(19) United States
(12) Patent Application Publication
(10) Pub. No.: US 2022/0108769 A1
(43) Pub. Date: Apr. 7, 2022

(54) METHODS FOR CHARACTERIZING THE LIMITATIONS OF DETECTING VARIANTS IN NEXT-GENERATION SEQUENCING WORKFLOWS

(71) Applicant: Sophia Genetics S.A., Saint Sulpice (CH)

(72) Inventors: Jonathan BIELER, Saint-Sulpice (CH); Christian POZZORINI, Saint-Sulpice (CH); Alex TUCK, Saint-Sulpice (CH); Zhenyu XU, Saint-Sulpice (CH)

(73) Assignee: Sophia Genetics S.A., Saint-Sulpice (CH)

(21) Appl. No.: 17/492,601

(22) Filed: Oct. 2, 2021

(30) Foreign Application Priority Data
Oct. 2, 2020 (EP) 20199741

(51) Int. Cl. G16B 30/10; G16B 5/20; G16B 20/20; G16B 40/20; C12Q 1/6869

(52) U.S. Cl. CPC G16B 30/10; G16B 5/20; C12Q 1/6869; G16B 40/20; G16B 20/20

(57) ABSTRACT

A genomic data analyzer may process the next generation sequencing data of a patient sample to identify whether a variant is present (positive variant calling), absent at a high confidence (negative variant calling), or equivocal (possible false negative calling) as falling under a calculated limit of detection (LOD). This LOD estimate corresponds the lowest variant allele fraction (VAF) detectable at the required sensitivity (true positive rate). The presently disclosed genomic data analyzer may improve any legacy variant caller by automatically calculating the limitations of variant calling detection for a user-defined sensitivity and minimal VAF of interest for any variant genomic position and/or mutation, depending on analytical factors of the NGS assay and workflow such as the sample type, the DNA sample amount and the NGS assay library conversion rate (LCR), and/or its molecular barcoding capability, as well as its NGS assay error profile.

### CROSS REFERENCE TO RELATED APPLICATIONS

This application claims the benefit of E.U. Provisional Application No. EP20199741, the entire contents of which are incorporated by reference herein.

### FIELD OF THE INVENTION

Methods described herein relate to genomic analysis in general, and more specifically to the use of genomic information for detecting and characterizing genomic variants.

### BACKGROUND OF THE INVENTION

Next-generation sequencing (NGS) assays are central to many branches of precision medicine, including liquid biopsies, tumor profiling and immunotherapy. However, translation to the clinic is impeded by reproducibility issues arising from unavoidable technical limitations. Regulatory and professional bodies are therefore calling for urgent measures to report NGS limitations so that errors are foreseen.

Clinical next-generation sequencing (NGS) assays present great opportunities for precision medicine as they can screen thousands of genomic loci for disease relevant alterations. Indeed, we are in the midst of a boom in which NGS technologies are being developed for diverse applications including pan-cancer profiling, liquid biopsies, biomarker discovery, immune profiling and personalized cancer vaccines (Chuah & Chew, 2020, J. Immunother. cancer 8, e000363; Zviran et al., 2020, Nat. Med. 26(7): 1114-1124; Malone et al., 2020, Genome Med. 12, 8). Despite their huge potential, recent studies found that NGS assays may suffer from significant reproducibility issues (Stetson et al., 2019, JCO Precis. Oncol. 19; Paweletz et al., 2019, JCO Precis. Oncol. 13; Kuderer et al., 2017, JAMA Oncol. 3, 996-998). This is caused by incorrect calls or miscalls (i.e. false positives and false negatives) occurring more often than expected. For example, a large-scale evaluation of NGS databases estimates the false negative rate to be 40-45% (Kim et al., 2019, PLoS One 14, e0222535). This problem is particularly acute for NGS technologies encountering low frequency genomic alterations, as is the case with early or non-invasive disease monitoring. For example, circulating tumor DNA (ctDNA) profiling is a minimally invasive approach capturing the heterogeneous and evolving nature of tumors (Heitzer et al., 2019, Nat. Rev. Genet. 20) that relies on detecting variants down to 0.1-0.3% (Matsumoto et al., 2020, Lung Cancer 139, 80-88; Jiang et al., 2019, Mol Med Rep 20, 593-603). These low variant allele frequencies (or equivalently, variant allele fractions) (VAFs) arise because ctDNA accounts for only a minority of cell-free DNA (cfDNA) in the blood (Heitzer et al., 2020, Trends Mol. Med. 26, 519-528; Sun et al., 2015, PNAS 112, E5503 LP-E5512). Despite claims that NGS assays can detect VAFs of <1% (Paweletz et al., 2019, supra; Chien et al., 2017, J. Clin. Oncol. 35, e23065-e23065; Plagnol et al., 2018, PLoS One 13, e0193802), a recent study found that when identical ctDNA samples were sent to different commercial laboratories, up to 90% of the low VAF variants were overlooked. This resulted in a high discordance between the results obtained by the different laboratories (Stetson et al., 2019, supra).

These reproducibility issues and high false negative rates are a major problem for clinical and research communities, as they undermine confidence in new technologies, cloud the results of clinical trials and can ultimately lead to incorrect treatment decisions. Discrepancies between NGS assays are often attributed to biological factors such as tumor heterogeneity (Kuderer et al., 2017, supra). In addition, and as showed by, Stetson et al., most testing errors arise from technical factors such as background noise or assay-specific nucleotide biases (Stetson et al., 2019, supra). This revelation recently sparked intense debate among clinicians, the FDA and other professional bodies (U.S. Food and Drug Administration, 13 Apr. 2018, Considerations for Design, Development, and Analytical Validation of Next Generation Sequencing Based In Vitro Diagnostics Intended to Aim in the Diagnosis of Suspected Germline Diseases; Weber et al., 2020, Cancers (Basel). 12, 1588), as it shows we have grossly underestimated the influence of technical factors and are unknowingly exceeding the capabilities of NGS assays. It also highlights the urgent need for addressing these technical limitations and its potential for a dramatic improvement in the reliability of NGS testing.

An obvious solution is to develop NGS assays with fewer technical limitations and, as detecting a genomic alteration relies on distinguishing signal from noise, research efforts have targeted both of these elements. For example, digital error suppression and barcoding strategies dramatically reduce background noise, while amplicon- or capture-based enrichment boosts signal across specific genomic regions (Newman et al., 2016, Nat. Biotechnol. 34, 547-555). Despite tremendous progress, there are some fundamental limitations that cannot be overcome, such as how much patient material can be sampled or stochastic sampling noise. Furthermore, for applications like early disease detection, there will always be a need to detect the lowest possible VAFs, so NGS assays will continually be pushed to their limits. Therefore, although NGS testing errors can be reduced, they cannot be eliminated, meaning that assay improvements are only part of the solution. Recognizing this, regulatory and professional bodies, clinicians and research communities are demanding that assay limitations are accurately and transparently reported alongside NGS results (U.S. Food and Drug Administration, 13 Apr. 2018, Considerations for Design, Development, and Analytical Validation of Next Generation Sequencing-Based In Vitro Diagnostics Intended to Aim in the Diagnosis of Suspected Germline Diseases; Merker et al., 2018, J Clin. Oncol. 36, 1631-1641; Tack et al., 2018, J Mol. Diagnostics 20, 743-753). The idea is not to eliminate testing errors, but to foresee them and act accordingly. At the same time, this increases confidence in the remaining results. Together with assay improvements, this elegant solution could eliminate most incorrect results.

Essentially, this strategy requires evaluating the likelihood that each positive or negative call is correct. For positive calls this is relatively straightforward, as sources of errors are well understood, and positive calls usually limited. Retesting is therefore feasible at a reasonable cost for all positive results deemed to be ambiguous, even if we are overly conservative. The situation is more complex for negative calls, which usually run into the thousands. Ideally, for each “negative” position the limit of detection (LOD) may be reported. This is the lowest VAF detectable at the required sensitivity (true positive rate). Knowing the LODs would reveal sites where a variant might not have been detected due to technical limitations. These potential false negatives could then be retested, focusing on hotspots for actionable mutations. Unfortunately, there is currently no effective way to calculate the LOD for every position interrogated by an NGS assay. This is because experimental approaches are limited to a handful of variants (leading to the practice of reporting a global LOD), and in silico predictions are held back by a lack of understanding of how LOD is determined (likely by many variables acting in combination). Previous modeling approaches either omit some variables, are limited to specific assays, require specialized controls, such as for instance molecular barcoding, do not estimate the LOD at different genomic positions or cannot be easily integrated into a diverse array of full, versatile, adaptive bioinformatic workflows (Blomquist et al., 2015, Biomol. Detect. Quantif 5, 30-37; Petrackova et al., 2019, Front. Oncol. 9, 1-6; Ma et al., 2019, Genome Biol. 20, 50; Xu et al., 2017, BMC Genomics 18, 5). An accurate and universal method to determine LOD at different genomic positions for a diversity of genomic analysis workflows and to report it alongside NGS results is therefore a key unresolved challenge that must be overcome to improve the reliability and reproducibility of genetic testing. In an embodiment, such a method shall be universal enough to be integrated into any data-driven medicine bioinformatic system and be compatible with the analysis of available NGS assays. In an embodiment, this method shall support an automated position-specific LOD estimation depending on the actual choice of the wetlab NGS assay.

In summary, there is a critical need to integrate the identification, characterization and reporting of NGS assay limitations into NGS workflows routinely used in clinical practice and research. This is key to improving the reliability of NGS testing, as it is now clear that miscalls (i.e. false positives and false negatives) are a persistent issue that cannot be resolved solely by technological improvements to NGS assays (which has been the focus of most prior art approaches). All NGS technologies have technical limitations, and there may always remain applications in which NGS technologies must operate near their limits. Thus, improved identification and reporting of assay limitations may be a way to satisfactorily address the incorrect variant calls that will inevitably occur. This capability needs to be integrated into automated NGS workflows that are cost effective, easy to operate, and suitable for routine clinical practice. Such workflows should be able to process data from different patient sample types, NGS technologies and variant types, account for the different sequencing error profiles that will be encountered, and operate in line with the requirements of the end user (e.g. required variant calling sensitivity). Furthermore, as the range of NGS technologies in routine use expands, these limitation-aware workflows must be easily adjustable and continue to meet the requirements of the assay and end user (e.g. the range of VAFs encountered). Preferably, initial setup would involve a consistent, robust and straightforward process, and the end user would not need to understand the intricacies of the NGS assay(s) and computational analyses. By identifying and reporting assay limitations, these workflows would enable the end user to make an informed decision as to whether further genomic analysis is required, and if so, for which genomic positions and variant types.

### BRIEF SUMMARY OF AN EMBODIMENT

A method is proposed for estimating a limit of detection (LOD) when characterizing a nucleic acid sequence variant (chr,pos,alt,ref) in data generated by a next-generation-sequencing (NGS) assay from a patient sample, the method comprising obtaining alignment data, relative to a reference genome, from the patient sample NGS data; identifying from the alignment data, with a variant caller, that said variant does not have a positive call status, obtaining a measurement of the molecular count for the patient sample at the genomic position (chr,pos) of said variant; and obtaining one or more analytical factors of the NGS assay used to process the patient sample. The method may further comprise producing, with a statistical model, synthetic alignment data for one or more simulated VAFs as a function of the measured molecular count and the analytical factors of the NGS assay; and estimating, from the synthetic alignment data, the detection sensitivity of said variant caller as a function of one or more of the simulated VAFs, for said assay, said DNA sample and said variant (chr, pos, ref, alt).

In an embodiment, the NGS assay used to process the patient sample is identified by an NGS assay identifier and wherein the one or more analytical factors of the NGS assay are predetermined values stored in a memory in association with the NGS assay identifier. In a further embodiment, the method may comprise obtaining a user-defined minimal variant allele fraction of interest (mVAF) for said variant; obtaining a user-defined desired sensitivity for calling said variant as positive; estimating, from the estimated detection sensitivity function, the sensitivity for said mVAF; and classifying the variant status as negative or equivocal as a function of said desired and estimated sensitivity. In another embodiment, the method may further comprise obtaining a user-defined desired sensitivity for calling said variant as positive; and estimating, from the estimated detection sensitivity function, a limit of detection LODₑₛₜ value as the lowest simulated VAF detectable with a sensitivity larger or equal to the user-defined sensitivity. The method may further comprise reporting the estimated limit of detection LODₑₛₜ value.

In an embodiment, the method may further comprise obtaining a user-defined minimal variant allele fraction of interest (mVAF) for said variant; obtaining a user-defined desired sensitivity for calling said variant as positive; estimating, from the estimated detection sensitivity function, a limit of detection LODₑₛₜ value as the lowest simulated VAF detectable with a sensitivity larger or equal to the user-defined sensitivity; and if the mVAF for said variant is larger or equal to the LODₑₛₜ, classifying the variant status as negative, otherwise classifying the variant status as equivocal. The method may further comprise reporting the variant status.

In an embodiment, the NGS assay features a molecular identifier, further comprising estimating the molecular count in the NGS sequencing data according to a molecular barcoding measurements in the alignment data. The method may further comprise measuring, in the alignment data, a total coverage at the genomic position (chr,pos) of said variant and producing, with the statistical model, the synthetic alignment data for one or more simulated VAFs as a function of the coverage. In an embodiment, the NGS assay does not feature a molecular identifier and the analytical factors of the NGS assay comprise a library conversion rate (LCR) profile, further comprising obtaining a DNA sample amount measurement; and estimating the molecular count in the library as a function of the DNA sample amount and a LCR value for said genomic position (chr, pos) in the LCR profile.

In an embodiment, the LCR profile is selected from the group consisting of a constant value for all genomic positions or a table of the library conversion rate value at each genomic position or set of positions. The LCR profile may depend on a DNA sample type. In an embodiment, the analytical features of the NGS assay comprise an NGS assay error profile selected from the group consisting of a constant value for all variants, a table of the error rate value at each variant position (chr,pos) or set of positions, a table of the error rate value for each variant mutation type (alt,ref) or set of variant mutations, or a table of the error rate value for each variant (chr,post,ref,alt). The NGS assay error profile may depend on a DNA sample type. In an embodiment, the producing, with a statistical model, synthetic sequencing data for different simulated VAFs comprises producing for each simulated VAF value one or more BAM files or different NGS data alignment features. The statistical model may be selected from the group consisting of a machine learning generative model or a biophysical generative model.

### BRIEF DESCRIPTION OF THE DRAWINGS

FIG. 1 represents a next generation sequencing system according to certain embodiments of the present disclosure.

FIG. 2 represents an improved genomic analysis system according to certain embodiments of the present disclosure.

FIG. 3 represents an improved variant calling workflow according to certain embodiments of the present disclosure.

FIG. 4 shows comparison of ctDNA reference materials as detailed in Example 1. Commutability of sonicated gDNA and rsctDNA, with clinical ctDNA, was assessed using assay A3. FIG. 4A: Collision rate, defined as the number of mapping positions at which more than one molecule (defined using molecular barcodes) aligned divided by the total number of mapping positions occupied in the data, plotted for each input type and amount. Error bars indicate the standard deviation across different genomic regions. FIG. 4B: Fragment size distribution. FIG. 4C: Distribution of error rates at genomic positions (n=6400) targeted by all assays. FIG. 4D: Average number of unique molecules covering each interrogated genomic position. Error bars indicate the standard deviation across different genomic regions.

FIG. 5 shows that LOD is determined at multiple scales. FIG. 5A: Experimental design as described in Example 1. FIG. 5B: True positive and false negative calls and measured VAFs from the experiment shown in A. In the absence of signal, VAF is set to 0.01%. FIG. 5C: Individual variant calls from ‘A’ arranged by VAF (individual columns), NGS assay (vertical groups), input material amount (horizontal groups) and rsctDNA dilutions (pairs of rows). The VAF for individual variants is indicated by the grey boxes of various shades (“VAF”), and for each NGS assay, variants are sorted by the number of true positives (which are summed in the lower plots). FIG. 5D: Library conversion rate (top), relative sequencing coverage (middle) and background noise (bottom) of each NGS assay shown as a function of genomic region. Arrows show the genomic positions of the 13 confirmed variants in ‘C’. For visualization purposes, data were averaged within exons. Relative sequencing coverage was calculated by averaging the mean-normalized coverage profiles observed in multiple samples. Error rate profiles show the mean of the statistical distribution (A1-A2: beta-binomial, A3: binomial) fitted to the number of artefactual base calls observed in multiple samples.

FIG. 6 shows investigating factors that determine variant calling sensitivity. FIG. 6A: Sensitivity (true positive count/(true positive count+false negative count)) measured for each NGS assay as a function of expected VAF. For each assay, an overall LOD (vertical line) was measured by fitting the data with a sigmoidal function (solid line), requiring a sensitivity of >90%. FIG. 6B: Variant calling concordance for FIG. 5B, defined as the fraction of confirmed SNVs having the same variant calling status in all NGS assays. The three bars show the concordance computed using all confirmed SNVs (n=234), SNVs with VAF >1% (n=30) and SNVs with VAF <1% (n=204). FIG. 6C: Sensitivity of NGS assays A1 and A3 when using various dilutions of rsctDNA (left) or sonicated gDNA (right). FIG. 6D: Sensitivity of each NGS assay plotted against input rsctDNA amount, using the data presented in FIG. 5. Error bars represent one standard deviation of theoretical uncertainty given the number of variants used to measure sensitivity. The light grey line shows the maximum theoretical sensitivity given the amount of starting material used. FIG. 6E: Fraction of genomic positions with error rates greater than or equal to a specific level (x-axis) measured for each NGS assay. FIG. 6F and FIG. 6G: Inferring the library conversion rate of NGS Assay A1 from variant calling results. FIG. 6F: The variant calling results shown in FIG. 5 were used to measure sensitivity (grey dots) as a function of the expected number of mutated DNA molecules present in the original sample. Error bars represent one standard deviation of theoretical uncertainty expected given the finite number of variants used to measure sensitivity. Data were fitted with a theoretical model (grey line) describing sensitivity as a function of the number of mutated molecules expected in the original sample and library conversion rate. The grey line shows the theoretical sensitivity computed using the maximum a posteriori estimation of library conversion rate. FIG. 6G: Posterior distribution of library conversion rate obtained by fitting a theoretical model to the data in FIG. 6F. FIG. 6H: Library conversion rate estimation for the NGS assays, calculated by dividing the observed number of molecules in the NGS data presented in FIG. 5 by the number expected given the amount of input DNA used (assays A2 and A3) or using the approach shown in FIG. 6F and FIG. 6G (assay A1).

FIG. 7 shows LOD-aware variant calling framework. FIG. 7A: Overview of the LOD-aware variant calling framework, highlighting how it enhanced the standard variant calling approach. In the standard approach (top track, boxed), NGS libraries obtained from clinical cfDNA samples are sequenced, then the data are interpreted by a variant caller which outputs positive calls. Positions where variants are not called are not usually reported. The LOD-aware framework built on this approach by using an in silico statistical model (center, encapsulated in thick dashed lines) to predict LODs for every interrogated position (right, curved block arrow) that can be reported alongside all results (arrows marked (iv)). The model required several inputs. These included the LCR and an error model, which were calculated once when setting up a given assay (bottom, arrows marked (iii)). Additional model inputs were obtained for each sample, including the amount (mass, in nanograms) of DNA used and sequencing coverage, with the latter being measured from the NGS data (top, arrows marked (ii)). Note that sequencing coverage and error rate were defined for each genomic position. After receiving these inputs, the model (arrows marked (i)) simulated a dilution experiment for each variant of interest by producing synthetic NGS data as follows. First, the model used the input DNA amount to estimate the number of ctDNA molecules covering a given genomic locus. Then, for a range of VAFs, the entire NGS workflow was simulated, including library preparation (ctDNA molecules were incorporated according to the LCR), PCR amplification and sequencing (reads were sampled to the measured sequencing depth, and errors injected based on the measured error rate). Short horizontal grey lines represent DNA fragments in the sample, amplified library and sequencing data. Black rectangles and grey triangles represent DNA mutations (at the position indicated by the vertical dashed line) and artefactual base calls, respectively. The model accounted for sampling variability (coin flip icons) during DNA sample generation, library preparation and sequencing. The output from the model (a series of VAFs and ref (N) and alt (N_{ALT}) read counts) was then presented to the variant caller (light grey), which called variants for each simulated read set and constructed a curve of estimated sensitivity as a function of VAF (dark grey curved block arrow). The predicted LOD could then be read off of this curve and was defined as the lowest VAF that could be detected with a desired sensitivity. Finally, the set of predicted LODs could be used to classify positions at which no variant was detected as either negative (the LOD is sufficiently low that a variant would have been detected if present) or equivocal (a false negative cannot be ruled out). FIG. 7B: The output obtained by performing LOD-aware variant calling on the rsctDNA dataset in FIG. 5. The observed VAF, input amount, variant call using a standard approach, predicted LOD and LOD-aware variant call is shown (top to bottom) for each confirmed variant, dilution and input amount (columns). Each pair of consecutive rows shows the results of replicates tested with NGS assays A1, A2 and A3, and variants are sorted by confirmed VAF and input amount. LOD-aware variant calls are defined as follows: positive—the variant is identified by the variant caller, negative—the variant is not identified by the variant caller and the VAF of interest is greater than or equal to the predicted LOD, equivocal—the variant is not identified by the variant caller and the VAF of interest is lower than the LOD. The “VAF of interest” is defined as that determined by dPCR, which was used to mimic clinical insight into the minimum expected VAF. FIG. 7C: LOD-aware variant calling status and measured VAF for each variant present in the rsctDNA samples. Note that “negative” calls are labeled as “false negative”, as in these cases, a confirmed variant was not detected even though the predicted LOD is sufficient.

FIG. 8 shows validation of the LOD-aware variant calling framework for rsctDNA samples. FIG. 8A: Schematic representation of the generative model used for NGS assay A3. For a given VAF and observed number of duplex molecules M, the statistical model described the number of molecules supporting the variant M_{ALT}. Grey lines represent DNA fragments in the sample and sequencing data. Black rectangles and grey triangles represent DNA mutations and artefactual base calls, respectively. FIG. 8B and FIG. 8C: Variant calls shown in FIG. 7B are grouped by predicted sensitivity (x-axis), and the true positive (TP) count, false negative (FN) count and sensitivity (TP/(TP+FN)) plotted for each group. Error bars indicate one standard deviation of theoretical uncertainty given the number of variants used to measure sensitivity. The diagonal grey line shows the ideal case, where observed sensitivity is equal to predicted sensitivity. FIG. 8B shows the results aggregated for all NGS assays, whereas FIG. 8C shows each assay individually. FIG. 8D: Variant calling concordance between the three NGS assays, based on FIG. 7C and excluding equivocal calls from the calculation.

FIG. 9 shows LOD-aware variant calling for 580 clinical cfDNA samples. FIG. 9A: VAF and input material amount for variants identified using dPCR (ddPCR and BEAMing) for two sets of clinical NSCLC cfDNA samples. Points are colored by the variant that they correspond to. The light grey line corresponds to VAFs and input materials for which, assuming an LCR of 100%, one molecule supporting the variant is expected. FIG. 9B: Variant calling status and dPCR and NGS VAF measurements for the confirmed variants from ‘A’ after testing using NGS assay A1 (for set #1) and A2 (for set #2). Extra positives are variants detected by NGS but not dPCR, and variants not detected (ND) by NGS are indicated with an arrow (and placed at the bottom edge of the plot). FIG. 9C: Output obtained by performing LOD-aware variant calling on the clinical datasets. The observed VAF, input amount, variant call using a standard approach, predicted LOD, and LOD-aware variant call are shown for each confirmed variant. LOD-aware variant calls were defined using 1% as the “minimal VAF of interest” (i.e. the lowest VAF we need to reliably detect). Using this 1% threshold, divided were non-positive positions into “negative” (LOD<1%) and “equivocal” (LOD>1%) classes. FIG. 9D: LOD prediction and LOD-aware variant calling status evaluated by comparing to the ground truth (dPCR status). Here included are dPCR-confirmed variants (shown in ‘C’) as well as all dPCR-validated negative cases for the four tested positions in the 580 samples. Positions with an LOD-aware “positive” label are classed as “true positive” (TP) when there is a dPCR-confirmed variant, and “false positive” (FP) when there is not. Positions with an LOD-aware “negative” label are classed as “true negative” (TN) if no variant was detected by dPCR (or a variant was detected by dPCR with VAF<1%), and “false negative” (FN) if there is a dPCR-confirmed variant with VAF>1%. Equivocal (E) calls retain the label “equivocal” and are positions we would exclude from subsequent analyses due to assay insufficiencies. For clinical cfDNA set #1, n=924, TP=44, FP=4, TN=607, FN=0, E=269, concordance=99.4%. For clinical cfDNA set #2, n=696, TP=53, FP=2, TN=352, FN=1, E=288, concordance=99.3%. Combined concordance (set #1 and #2)=99.3%.

FIG. 10 shows validation of the sensitivity predicted by LOD-aware variant calling for clinical samples. Data shown in FIG. 9C were analyzed to verify the agreement between predicted and observed sensitivity (black dots). Variant calls were grouped according to their predicted sensitivity. For each group, observed sensitivity (black dots) was computed by counting the number of true positives (TP count, top histogram) and false negatives (FN count, bottom). The error bars indicate the standard deviation of the posterior distribution of the observed sensitivities.

FIG. 11 shows the LOD landscape for clinically relevant variants. FIG. 11A: Predicted LODs for 89 variants annotated as pathogenic in ClinVar are displayed on a heatmap, with each column corresponding to one clinical sample tested with NGS assay A1 (n=360). Essentially, this demonstrates the lowest variant fraction that could be detected at each site in each sample, thus revealing our potential to identify variants. Rows and columns are sorted according to the average LOD computed across samples and variants, respectively. Top: the average LOD for each clinical sample, across all variants. Right: the average LOD for each variant, across all samples. Error bars indicate 5% and 95% quantiles. Left and bottom: “LOD influence” is an analysis to determine which of the three model inputs (background noise, coverage depth or input amount) has the most influence on LOD, and thereby which could be adjusted to improve the LOD. This is evaluated by calculating the increase in predicted sensitivity (at the current LOD) when one of the three model inputs is adjusted (improved) by 10%. The values associated with changing each input are averaged across samples (left) or variants (bottom), then normalized by dividing by the sum of the three average values (as described in Sensitivity analysis). Thus, the length of each colored line represents the relative gain in sensitivity that would be achieved by improving each of the input factors. FIG. 11B: Predicted LODs, limiting factors and summary statistics for the second set of clinical cfDNA samples, processed with NGS assay A2. Data are presented as in ‘A’.

FIGS. 12A-12B show the comparison of the LOD-aware variant calling framework for rsctDNA samples between in-house (the proprietary variant caller of the Sophia Genetics DDM genomic analysis platform) and VarDict variant calling algorithms.

FIG. 13 shows the detection status and predicted LOD for 447 confirmed variants in cancer reference ctDNA mixtures processed using the Illumina TruSight Tumor 170 assay and the proprietary variant caller of the Sophia Genetics DDM genomic analysis platform. Three replicates were included (different libraries prepared by the same laboratory). Top: VAFs. Bottom: observed and predicted sensitivities for these variants.

FIG. 14 illustrates a block diagram of a distributed computer system that can implement one or more aspects of an embodiment of the present invention.

FIG. 15 illustrates a block diagram of an electronic device that can implement one or more aspects of an embodiment of the invention.

### DETAILED DESCRIPTION

The present disclosure may be based, at least in part, on the fact that the presently disclosed genomic data analyzer may process the next generation sequencing data of a patient DNA sample to identify whether a variant is present (positive variant calling), absent at a high confidence (negative variant calling), or equivocal (possible false negative calling) as falling under a calculated limit of detection (LOD). This LOD estimate may correspond to the lowest variant allele fraction (VAF) detectable at the required sensitivity (true positive rate). The presently disclosed genomic data analyzer may improve any legacy variant caller by automatically accounting for the limitations of variant calling detection for a user-defined sensitivity and minimal VAF of interest for any variant genomic position and/or mutation depending on analytical factors of the NGS assay and workflow such as the sample type; the DNA sample amount and the NGS assay library conversion rate (LCR), or its molecular barcoding capability; as well as its NGS assay error profile.

### Definitions

A “DNA sample” refers to a nucleic acid sample derived from an organism, as may be extracted for instance from a solid tumor or a fluid. The organism may be a human, an animal, a plant, a fungus, or a microorganism. The nucleic acids may be found in a solid sample such as a Formalin-Fixed Paraffin-Embedded (FFPE) sample. Alternately, the nucleic acids may be found in a liquid biopsy, possibly in limited quantity or low concentration, such as for instance circulating tumor DNA in blood or plasma.

A “DNA fragment” refers to a short piece of DNA resulting from the fragmentation of high molecular weight DNA. Fragmentation may have occurred naturally in the sample organism, or may have been produced artificially from a DNA fragmenting method applied to a DNA sample, for instance by mechanical shearing, sonication, enzymatic fragmentation and other methods. After fragmentation, the DNA pieces may be end repaired to ensure that each molecule possesses blunt ends. To improve ligation efficiency, an adenine may be added to each of the 3′ blunt ends of the fragmented DNA, enabling DNA fragments to be ligated to adaptors with complementary dT-overhangs.

A “DNA product” refers to an engineered piece of DNA resulting from manipulating, extending, ligating, duplicating, amplifying, copying, editing and/or cutting a DNA fragment to adapt it to a next-generation sequencing workflow.

A “DNA-adaptor product” refers to a DNA product resulting from combining a DNA fragment with a DNA adaptor to make it compatible with a next-generation sequencing workflow. Different approaches exist to combine a DNA fragment and a DNA adaptor. These include amplicon-based protocols or capture based protocols, or hybrid protocols.

A “DNA library” refers to a collection of DNA products or DNA-adaptor products that adapt DNA fragments for compatibility with a next-generation sequencing workflow.

A “DNA amount” refers to the quantity of purified DNA present in the sample that is processed with a NGS assay. DNA amount is usually measured in nanograms or micrograms. DNA amount can also be measured in units of human genome equivalents or haploid human genome equivalents.

A “nucleotide sequence” or a “polynucleotide sequence” refers to any polymer or oligomer of nucleotides such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string). It may be DNA or RNA, or a combination thereof. It may be found permanently or temporarily in a single-stranded or a double-stranded form. Unless otherwise indicated, nucleic acids sequences are written left to right in 5′ to 3′ orientation.

“Ligation” refers to the joining of separate double stranded DNA sequences. The latter DNA molecules may be blunt ended or may have compatible overhangs to facilitate their ligation. Ligation may be produced by various methods, for instance using a ligase enzyme, performing chemical ligation, and other methods.

“Amplification” refers to a polynucleotide amplification reaction to produce multiple polynucleotide sequences replicated from one or more parent sequences. Amplification may be produced by various methods, for instance a polymerase chain reaction (PCR), a linear polymerase chain reaction, a nucleic acid sequence-based amplification, rolling circle amplification, and other methods.

“Sequencing” refers to reading a sequence of nucleotides as a string. High throughput sequencing (HTS) or next-generation-sequencing (NGS) refers to real time sequencing of multiple sequences in parallel, typically between 50 and a few thousand base pairs per sequence. Exemplary NGS technologies include those from Illumina, Ion Torrent Systems, Oxford Nanopore Technologies, Complete Genomics, Pacific Biosciences, BGI, and others. Depending on the actual technology, NGS sequencing may require sample preparation with sequencing adaptors or primers to facilitate further sequencing steps, as well as amplification steps so that multiple instances of a single parent molecule are sequenced, for instance with PCR amplification prior to delivery to flow cell in the case of sequencing by synthesis. In whole genome sequencing (WGS), DNA fragments originating from all genomic regions are sequenced. Depending on the NGS assay, only DNA fragments originating form one or more specific regions of interest (e.g., a particular gene of interest or all the exons present in the genome) are enriched and successfully sequenced. Enrichment can be performed using different technologies, including capture sequencing or amplicon sequencing. In this case, sequencing only targets parts of the entire genome. This approach is often referred to as targeted sequencing.

An “analytical factor of a NGS assay” or “analytical feature of a NGS assay” or “technical factors of a NGS assay” refers to the steps and components of the NGS assay. This covers all aspects of the experiment including DNA isolation, DNA quality, fragmentation, library preparation, enrichment method if relevant, type of barcodes and barcode ligation if relevant, library amplification and library sequencing. Examples of analytical factors of the NGS assay include, but are not restricted to, workflow error rate profile, Library Conversion Rate (LCR), and others. It is understood that different NGS assays and workflows may be characterized by different analytical factors.

A “Library Conversion Rate” (LCR) refers to the ratio between the number of molecules in the library divided by the theoretical number of molecules determined for the sample input DNA amount. The molecule number in a library is calculated based on the sequencing depth and the detected sequencing diversity (number of molecules). The LCR thus represents the percentage of input DNA molecules covering a genomic region of interest present in a DNA sample, which an NGS assay can successfully transform into sequence-able DNA products. In capture-based NGS assays, LCR can also refer to the percentage of input DNA molecules present in a DNA sample, for which at least one sequence-able DNA product was obtained after completion of the capture step.

A “sequencing error profile” or “error profile” or “workflow error profile” or “workflow error rate” refers to the list of parameters describing the rate at which artefactual base calls are introduced during the NGS workflow at each genomic position. The error profile accounts for different sources of artefacts, including PCR errors, sequencing errors and errors introduced at the read alignment step. The nature of the sequencing error profile depends on the nature of the statistical model using to generate synthetic alignment data. For example, if errors are modeled using a Beta-binomial distribution, the error profile may simply include two parameters defining the mean and the variance.

An “adapter” or “adaptor” refers to a short double-stranded or partially double-stranded DNA molecule of around 10 to 100 nucleotides (base pairs) which has been designed to be ligated to a DNA fragment. An adaptor may have blunt ends, sticky ends as a 3′ or a 5′ overhang, or a combination thereof. For example, to improve ligation efficiency, an adenine may be added to each of the 3′ blunt ends of the fragmented DNA prior to adaptor ligation, and the adaptor may have a thymidine overhang on the 3′ end to base-pair with the adenine added to the 3′ end of the fragmented DNA. The adaptor may have a phosphorothioate bond before the terminal thymidine on the 3′ end to prevent an exonuclease from trimming the thymidine, thus creating a blunt end when the end of the adaptor being ligated is double-stranded.

A “partially double stranded adaptor” refers to an adaptor including both a double-stranded region and a single stranded region. The double stranded region of the adaptor contains the ligation domain, whereas the single stranded region contains the primer sequences used for subsequent library amplification, barcoding and/or sequencing. The single stranded region can either be composed of two single stranded arms, a 5′ arm and a 3′ arm, as it is the case for so-called Y-shape adaptors, or the single stranded region of the partially double stranded adaptor can form a hairpin or a loop, as it is the case for so-called U-shape adaptors. The term partially double stranded adaptor refers thus both to Y-shape and U-shape adaptors or a combination thereof.

A group of “PCR duplicates” refers to a set of DNA products generated by PCR amplification from a single stranded DNA molecule belonging to a DNA-adaptor product derived from an original DNA fragment.

A “molecular identifier” or “molecular tag” or “molecular barcode” or “molecular code” refers to a feature of the DNA molecule that is used to identify PCR duplicates. Different types of molecular identifiers exist, including endogeneous UMIs (e.g., the mapping position of a DNA fragment) or exogenous UMIs (e.g., a random or pseudo-random sequence introduced in the DNA adaptor).

“Aligning” or “alignment” or “aligner” refers to mapping and aligning base-by-base, in a bioinformatics workflow, the sequencing reads to a reference genome sequence, depending on the application. For instance, in a targeted enrichment application where the sequencing reads are expected to map to a specific targeted genomic region in accordance with the hybrid capture probes used in the experimental amplification process, the alignment may be specifically searched relative to the corresponding sequence, defined by genomic coordinates such as the chromosome number, the start position and the end position in a reference genome. As known in bioinformatics practice, in some embodiments “alignment” methods as employed herein may also comprise certain pre-processing steps to facilitate the mapping of the sequencing reads and/or to remove irrelevant data from the reads, for instance by removing non-paired reads, and/or by trimming the adapter sequence at the end of the reads, and/or other read pre-processing filtering means.

“Coverage” refers to the number of sequencing reads that have been aligned to a genomic position or to a set of genomic positions. In general, a genomic region with a higher coverage is associated with a higher reliability in downstream genomic characterization, in particular when calling variants. In target enrichment workflows, only a small subset of regions of interest in the whole genome is sequenced and it may therefore be reasonable to increase the sequencing depth without incurring too significant data storage and processing overheads. In some genomic analysis applications not requiring a high resolution along the genome, for instance in detecting copy number alterations, low-pass (LP) coverage (1×-10×) or even ultra-low-pass (ULP) coverage (<1×—not all positions are sequenced) may be more efficient in terms of information technology infrastructure costs, but these workflows require more sophisticated bioinformatics methods and techniques to process the less reliable data output from the sequencer and aligner. Moreover, apart from the higher cost related to data storage and processing, the operational cost of an experimental NGS run, that is, loading a sequencer with samples for sequencing, also needs to be optimized by balancing the coverage depth and the number of samples which may be assayed in parallel in routine clinical workflows. Indeed, next generation sequencers are still limited in the total number of reads that they can produce in a single experiment (i.e. in a given run). The lower the coverage, the fewer reads per sample for the genomic analysis, and the higher the number of samples that can be multiplexed within a next generation sequencing run.

A “molecular count” or “count of molecules” refers to the number of distinct DNA molecules present in an original DNA sample for which at least one DNA product is observed in a DNA library or in alignment data.

“Variant calling” refers to identifying, in a bioinformatics workflow, actual sequence variants in the aligned reads relative to a reference sequence. In bioinformatics data processing, a variant is uniquely identified by its position along a chromosome (chr,pos) and its difference relative to a reference genome at this position (ref, alt). Variants may include single nucleotide permutations (SNPs) or other single nucleotide variants (SNVs), insertions or deletions (INDELs), copy number variants (CNVs), as well as large rearrangements, substitutions, duplications, translocations, and others. Preferably variant calling is robust enough to sort out the real sequence variants from variants introduced by the amplification and sequencing noise artefacts, for example. In a bioinformatics workflow, a variant caller may apply variant calling to produce one or more variant calls.

“Mutation” refers to a type of alteration in a nucleic acid sequence, such as a SNP, SNV or indel. A “variant” is a specific mutation occurring at a specific genomic position.

“Variant allele fraction” or “Variant allele frequency” or “VAF” is a measure of the fraction of DNA molecules in an original specimen carrying a variant. VAF can be measured in an NGS experiment by counting the number of sequencing reads that support a genomic variant divided by the overall coverage at that genomic position. Depending on the NGS assay, more sophisticated approaches may be used to measure VAF. For instance, when UMIs are used, PCR duplicates may be identified, and the VAF may be measured by counting the fraction of unique molecules supporting the variant, rather than the fraction of sequencing reads.

A “statistical model” is a mathematical model that assigns a probability to an instance of data generated by a stochastic process of interest. Statistical models can be divided in two subclasses: biophysical and machine learning models. A “biophysical statistical model” is a statistical model derived from first principles incorporating insights into the biophysical process underlying the data generation process. Model parameters usually have a biophysical interpretation as well as physical units. In contrast, a “machine learning statistical model” is a statistical model not entirely derived from first principles, but rather learnt from a training dataset using machine learning algorithms. In contrast to biophysical statistical models, machine learning statistical models can be derived without prior knowledge or understanding of the data generation process. The internal components and parameters of a machine learning model may not usually reflect biophysical processes and quantities.

A “generative model” is a probabilistic model that generates simulated data given a set of known parameters. Generative models can be divided in two subclasses: biophysical and machine learning models. A “biophysical generative model” is a generative model derived from first principles incorporating insights into the biophysical process underlying the data generation process. Model parameters usually have a biophysical interpretation as well as physical units. In contrast, a “machine learning generative model” is a generative model not entirely derived from first principles, but rather learnt from a training dataset using machine learning algorithms. In contrast to biophysical generative models, machine learning generative models can be derived without prior knowledge or understanding of the data generation process. The internal components and parameters of a machine learning model may not usually reflect biophysical processes and quantities.

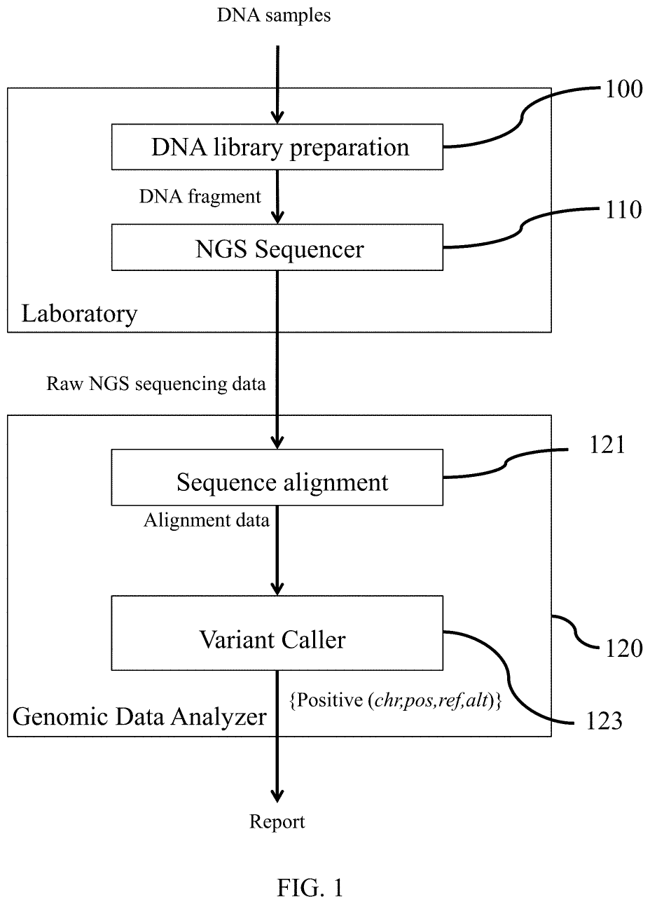

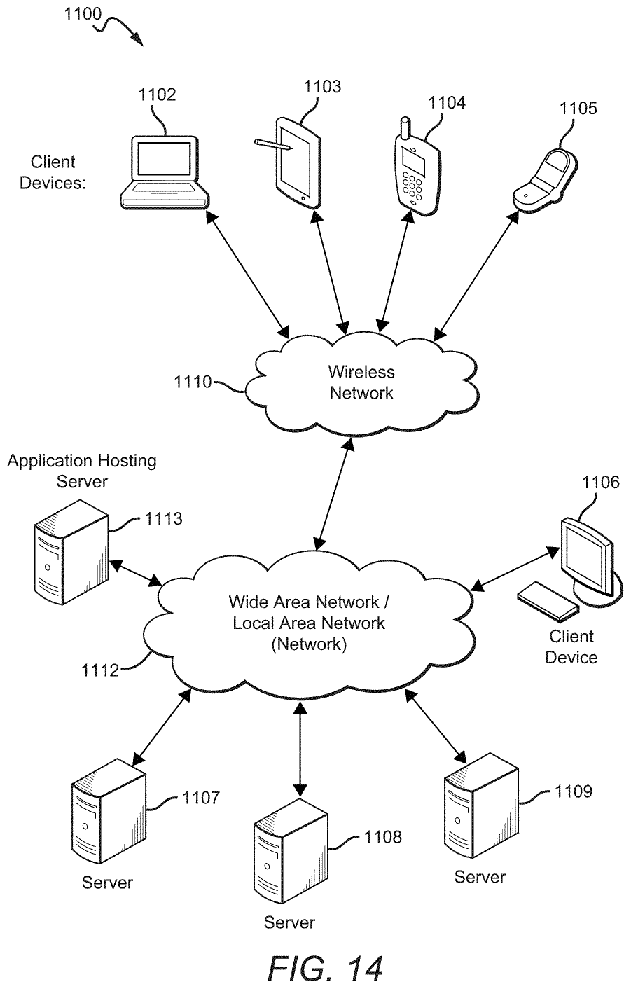

FIG. 14 illustrates components of one embodiment of an environment in which the invention may be practiced. Not all of the components may be required to practice the invention, and variations in the arrangement and type of the components may be made without departing from the spirit or scope of the invention. As shown, the system 1100 includes one or more Local Area Networks (“LANs”)/Wide Area Networks (“WANs”) 1112, one or more wireless networks 1110, one or more wired or wireless client devices 1106, mobile or other wireless client devices 1102-1105, servers 1107-1109, and may include or communicate with one or more data stores or databases. Various of the client devices 1102-1106 may include, for example, desktop computers, laptop computers, set top boxes, tablets, cell phones, smart phones, smart speakers, wearable devices (such as the Apple Watch) and the like. Servers 1107-1109 can include, for example, one or more application servers, content servers, search servers, and the like. FIG. 1 also illustrates application hosting server 1113.

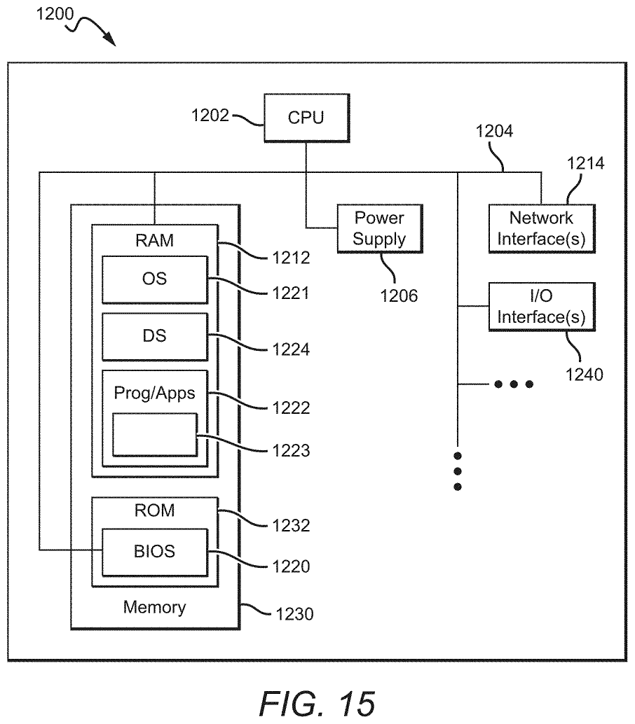

FIG. 15 illustrates a block diagram of an electronic device 1200 that can implement one or more aspects of an apparatus, system and method for adaptive parsing and processing of text to facilitate user engagement (the “Engine”) according to one embodiment of the invention. Instances of the electronic device 1200 may include servers, e.g., servers 1107-1109, and client devices, e.g., client devices 1102-1106. In general, the electronic device 1200 can include a processor/CPU 1202, memory 1230, a power supply 1206, and input/output (I/O) components/devices 1240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 1200. A touchscreen may determine whether a user is providing input by, for example, determining whether the user is touching the touchscreen with a part of the user's body such as his or her fingers. The electronic device 1200 can also include a communications bus 1204 that connects the aforementioned elements of the electronic device 1200. Network interfaces 1214 can include a receiver and a transmitter (or transceiver), and one or more antennas for wireless communications.

The processor 1202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 1230, which can include Random Access Memory (RAM) 1212 and Read Only Memory (ROM) 1232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 1221, data storage 1224, which may include one or more databases, and programs and/or applications 1222, which can include, for example, software aspects of the program 1223. The ROM 1232 can also include Basic Input/Output System (BIOS) 1220 of the electronic device.

Software aspects of the program 1223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 1206 contains one or more power components, and facilitates supply and management of power to the electronic device 1200.

The input/output components, including Input/Output (I/O) interfaces 1240, can include, for example, any interfaces for facilitating communication between any components of the electronic device 1200, components of external devices (e.g., components of other devices of the network or system 1100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 1240 and the bus 1204 can facilitate communication between components of the electronic device 1200, and in an example can ease processing performed by the processor 1202.

Where the electronic device 1200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 1102-1106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 1102-1106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MIMS, playing games (such as fantasy sports leagues), receiving advertising, watching locally stored or streamed video, or participating in social networks.

In example aspects of the apparatus, system and method implementing the Engine, one or more networks, such as networks 1110 or 1112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 1102-1106, which are communicatively coupled to servers including servers 1107-1109. Moreover, client devices 1102-1106 may be communicatively (wirelessly or wired) coupled to one another. In particular, software aspects of the Engine may be implemented in the program 1223. The program 1223 may be implemented on one or more client devices 1102-1106, one or more servers 1107-1109, and 1113, or a combination of one or more client devices 1102-1106, and one or more servers 1107-1109 and 1113.

### Genomic Analysis System

An exemplary genomic analysis system and workflow will now be described in further detail with reference to FIG. 1. As will be apparent to those skilled in the art of DNA analysis, a genomic analysis workflow comprises preliminary experimental steps to be conducted in a laboratory (also known as the “wet lab”) to produce DNA analysis data, such as raw sequencing reads in a next-generation sequencing workflow, as well as subsequent data processing steps to be conducted on the DNA analysis data to further identify information of interest to the end users, such as the detailed identification of DNA variants and related annotations, with a bioinformatics system (also known as the “dry lab”). Depending on the actual application, laboratory setup and bioinformatics platforms, various embodiments of a DNA analysis workflow are possible. FIG. 1 describes an example of an NGS system comprising a wet lab system wherein DNA samples are first experimentally prepared with a DNA library preparation protocol 100 which may produce, adapt for sequencing and amplify DNA fragments to facilitate the processing by an NGS sequencer 110. In a next generation sequencing workflow, the resulting DNA analysis data may be produced as a data file of raw sequencing reads, for instance in the FASTQ format. The workflow may then further comprise a dry lab Genomic Data Analyzer system 120 which takes as input the raw sequencing reads for a pool of DNA samples prepared according to the proposed methods, and applies a series of data processing steps to characterize certain genomic features of the input samples. An exemplary Genomic Data Analyzer system 120 is the Sophia Data Driven Medicine platform (Sophia DDM) as already used by more than 1000 hospitals worldwide in 2020 to automatically identify and characterize genomic variants and report them to the end user, but other systems may be used. Different detailed possible embodiments of data processing steps as may be applied by the Genomic Data Analyzer system 120 for genomic variant analysis are also possible.

As illustrated on FIG. 1, the Genomic Data Analyzer 120 may process the sequencing data to produce a genomic data analysis report by employing and combining different data processing methods. The Genomic Data Analyzer 120 may comprise a sequence alignment module 121, which compares the raw NGS sequencing data to a reference genome, for instance the human genome in medical applications, or an animal genome in veterinary applications. The sequence alignment module 121 may be configured to execute different alignment algorithms. Standard raw data alignment algorithms such as Bowtie2 or BWA that have been optimized for fast processing of numerous genomic data sequencing reads may be used, but other embodiments are also possible. The alignment results may be represented as one or several files in BAM or SAM format, as known to those skilled in the bioinformatics art, but other formats may also be used, such as compressed formats or formats optimized for order-preserving encryption and/or a combination thereof, depending on the genomic data analyzer 120 requirements for storage optimization and/or genomic data privacy enforcement.

The resulting alignment data may be further filtered and analyzed by a Variant Caller module 123 to retrieve variant information such as SNP and INDEL polymorphisms. The Variant Caller module 123 may be configured to execute different variant calling algorithms, either on the pre-processed consensus alignment data or directly on the alignment data for probabilistic variant callers, or on a set of pre-processed NGS data alignment features, as will be apparent to those skilled in the art of bioinformatics. Exemplary Variant Caller modules 123 which have been recently developed for somatic variant detection include, but are not limited to, Illumina Strelka2, GATK Mutect2, VarScan2, Shimmer, NeuSomatic, MuClone, MultiSNV, Needlestack, Qiagen smCounter or smCounter2, Sophia DDM probabilistic variant caller and others.

In prior art bioinformatics NGS workflows, the variants detected by the Variant Caller 123 are typically reported as having a “positive” variant calling status. The corresponding detected variant information may then be output by the Genomic Data Analyzer module 120 as a genomic variant report, for instance using the VCF (Variant Calling Format) file format, for further processing by the end user, for instance with a visualization tool, and/or by a further variant annotation processing module (not represented).

The Genomic Data Analyzer 120 may be a computer system or part of a computer system including a central processing unit (CPU, “processor” or “computer processor” herein), memory such as RAM and storage units such as a hard disk, and communication interfaces to communicate with other computer systems through a communication network, for instance the internet or a local network. Examples of genomic data analyzer computing systems, environments, and/or configurations include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, graphical processing units (GPU), and the like. In some embodiments, the computer system may comprise one or more computer servers, which are operational with numerous other general purpose or special purpose computing systems and may enable distributed computing, such as cloud computing, for instance in a genomic data farm. In some embodiments, the genomic data analyzer 120 may be integrated into a massively parallel system. In some embodiments, the genomic data analyzer 120 may be directly integrated into a next generation sequencing system.

The Genomic Data Analyzer 120 computer system may be adapted in the general context of computer system-executable instructions, such as program modules, being executed by a computer system. Generally, program modules may include routines, programs, objects, components, logic, data structures, and so on that perform particular tasks or implement particular abstract data types. As is well known to those skilled in the art of computer programming, program modules may use native operating system and/or file system functions, standalone applications; browser or application plugins, applets, etc.; commercial or open source libraries and/or library tools as may be programmed in Python, Biopython, C/C++, or other programming languages; custom scripts, such as Perl or Bioperl scripts.

Instructions may be executed in distributed cloud computing environments where tasks are performed by remote processing devices that are linked through a communications network. In a distributed cloud-computing environment, program modules may be located in both local and remote computer system storage media including memory storage devices.

### Genomic Data Analyzer

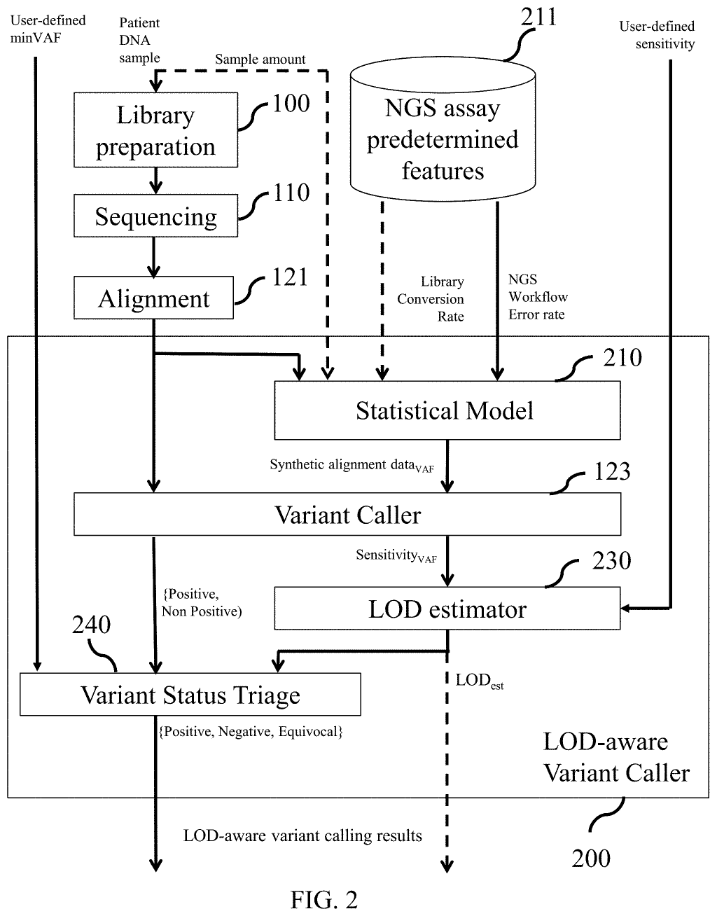

In an embodiment, the Genomic Data Analyzer system 120 may be adapted to produce an improved LOD-aware variant calling report from the analysis of alignment data in an NGS bioinformatics workflow. FIG. 2 shows a possible system architecture for an LOD-aware variant caller 200 as an improvement of a conventional variant caller module 123. In an embodiment, the LOD-aware variant caller 200 may be adapted to produce, with a statistical model 210, synthetic sequencing data and to automatically estimate from this synthetic sequencing data, with an LOD estimator module 230, a limit of detection for variant calling at a user-defined desired sensitivity. The proposed LOD-aware Variant Caller 200 may also classify, with a Variant Status Triage module 240, whether this variant is present (positive variant calling), absent at a high confidence (negative variant calling), or equivocal (possible false negative calling) for a user-defined minimum variant allele fraction (VAF) of interest.

In an embodiment, the LOD-aware Variant Caller 200 comprises a Variant Caller module 123 adapted to operate with an NGS assay comprising library preparation 100, NGS sequencing 110 and NGS alignment 121. Depending on the choice of the NGS assay and workflow, different analytical factors may characterize it, such as its workflow error rate profile. As will be apparent to those skilled in the art of NGS genomic analysis, the NGS workflow error rate may be constant, or may vary with the variant positions and/or mutations.

In a possible embodiment, it may be possible to infer the molecular count present in the NGS data after NGS sequencing by combining information about the sample DNA amount, the Library Conversion Rate (LCR) profile as an additional analytical factor characterizing the NGS assay, and the read coverage.

A count of molecules in the DNA library may be first estimated by converting the sample DNA amount in genome equivalents (GE) or haplotype genome equivalents (hGE), and by accounting for the number of molecules present in the sample DNA that the NGS assay could not convert into a DNA library. For example, in a situation where the sample DNA amount is 10 ng and LCR=50%, then the count of molecules present in the DNA library may be estimated as 300[hGE/ng]*10[ng]*0.5=1500 molecules. Since library preparation is a stochastic process, the count of molecules in the DNA library may also be estimated using a stochastic variable. For example, it may be described by a Poisson distribution with an expected value set at 300*ng*LCR.

The molecular count present in the NGS data can then be further estimated by combining the molecular count present in the DNA library and the coverage depth measured from the alignment file. The LOD-aware Variant Caller 200 may measure, in the alignment data, the total coverage at the genomic position (chr,pos) of said variant. In an embodiment, the total coverage value may be the sequencing read depth at the genomic position (chr,pos), but other embodiments are also possible, for instance only counting read depth of sufficient quality and other coverage measurement methods, as will be apparent to those skilled in the art of NGS bioinformatics. In cases where coverage depth is much larger compared to the DNA library molecular count, it is possible to assume that all molecules represented in the DNA library have been sequenced at least once. In this case, the count of molecules present in the DNA library provides a good estimation of the molecular count present in the NGS data. Otherwise, one has to consider that some molecules, while being present in the DNA library, may not be sequenced. In a possible embodiment, assuming that all molecule molecules represented in the DNA library are equally likely to be sequenced, the molecular count in the NGS data can be estimated as 300*ng*LCR*(1−exp(coverage/(300*ng*LCR)). In an alternate embodiment, since sequencing is a stochastic process, the molecular count in the NGS data may be described as a probability distribution with expected value equal to 300*ng*LCR*(1−exp(−coverage/(300*ng*LCR)) (e.g., Poisson distribution). Finally, in cases where the coverage depth is much smaller compared to the count of molecules in the DNA library, the number of unique molecules present in the NGS data tends to equal the coverage. In the latter scenario, the number of unique molecules present in the NGS data can thus be estimated simply by counting the number of reads in the NGS data.

In a possible embodiment, depending on its design, the NGS assay may enable the conversion of a patient sample DNA amount into a molecular amount depending on its Library Conversion Rate (LCR) profile as an additional, optional analytical factor characterizing the NGS assay. The LCR profile may be defined as a constant rate for all genomic positions or may vary with the genomic position. For example, the input molecular count may be calculated as 300*ng*LCR(chr,pos)*(1−exp(−coverage(chr,pos)/(300*ng*LCR(chr,pos)))), where 300*ng*LCR is the number of input DNA molecules which are converted into DNA product and successfully captured.

In an alternate embodiment, the NGS assay may employ an intrinsic molecular barcoding technology. Examples of molecular barcoding technologies include the use of Unique Molecular Identifiers (UMIs) which may be employed for consensus sequencing (Xu et al., 2018, supra). Examples of a Variant Caller 123 which exploits a molecular barcoding technology is described in “MAGERI: Computational pipeline for molecular-barcoded targeted resequencing”, Shugay et al., PLoS Comput. Biol. 2017 May; 13(5) or in “smCounter2: an accurate low frequency variant caller for targeted sequencing data with unique molecular identifiers”, Xu et al., Bioinformatics, Vol. 35(8), April 2019. An alternate numerical coding technology to UMI barcoding is also described in co-pending patent application PCT/EP2020/076246. An alternate method to estimate a molecular count may comprise counting similar reads using the start and end positions in the alignment data in the absence of mapping positions. Any of the above molecular barcoding methods, when integrated in an NGS assay, may enable to measure a molecular count for a variant directly from the alignment data.

In an embodiment, the LOD-aware Variant Caller 200 may be designed to operate with a diversity of NGS assay workflows, each NGS assay workflow being characterized by its analytical factors which can be defined once and stored as NGS assay pre-determined features 211 in a repository of the Genomic Data Analyzer system 120. The LOD-aware Variant Caller 200 may then retrieve the NGS assay pre-determined features 211 when analyzing a variant depending on the NGS assay which has been applied to the patient DNA sample to be analyzed.

In an embodiment, the NGS assay analytical factors may comprise at least an NGS workflow error profile, and optionally an LCR profile, to characterize an NGS assay and possibly a sample type, but other embodiments are also possible. The analytical factors may be predetermined values stored in a memory in association with an NGS assay identifier to facilitate their retrieval by the LOD-aware variant caller, but other embodiments are also possible.

The LCR profile may be a constant value for all genomic positions, or a table of the library conversion rate value at each genomic position (chr,pos) or at sets of genomic positions. The LCR profile may also depend upon the DNA sample type.

The NGS assay error profile may be a constant value for all variants, a table of the error rate value at each variant position (chr,pos) or sets of positions, or a table of the error rate value for each variant (chr,post,ref,alt) or sets of variant types (ref,alt). The NGS assay error profile may also depend upon the DNA sample type.

In an embodiment, as represented on FIG. 2, the LOD-aware Variant Caller 200 may comprise a Variant Caller module 123 to detect whether a variant has a positive or non-positive status, a Generative Model module 210 to generate in silico synthetic alignment data for different VAFs, an LOD estimator module 230 to estimate a limit of detection for the Variant Caller operating on a variant of interest to the user at a user defined sensitivity and minimal VAF for said variant, and a Variant Status Triage module 240 to further classify the variant status as positive, negative or equivocal. In one embodiment, the LOD-aware variant calling results may be displayed to the end user on a graphical user interface. In another possible embodiment, the LOD-aware variant calling results may be produced as a text file, for instance as an extension of the VCF file format for further automated processing. Other embodiments are also possible.

### LOD-Aware Variant Calling Workflow

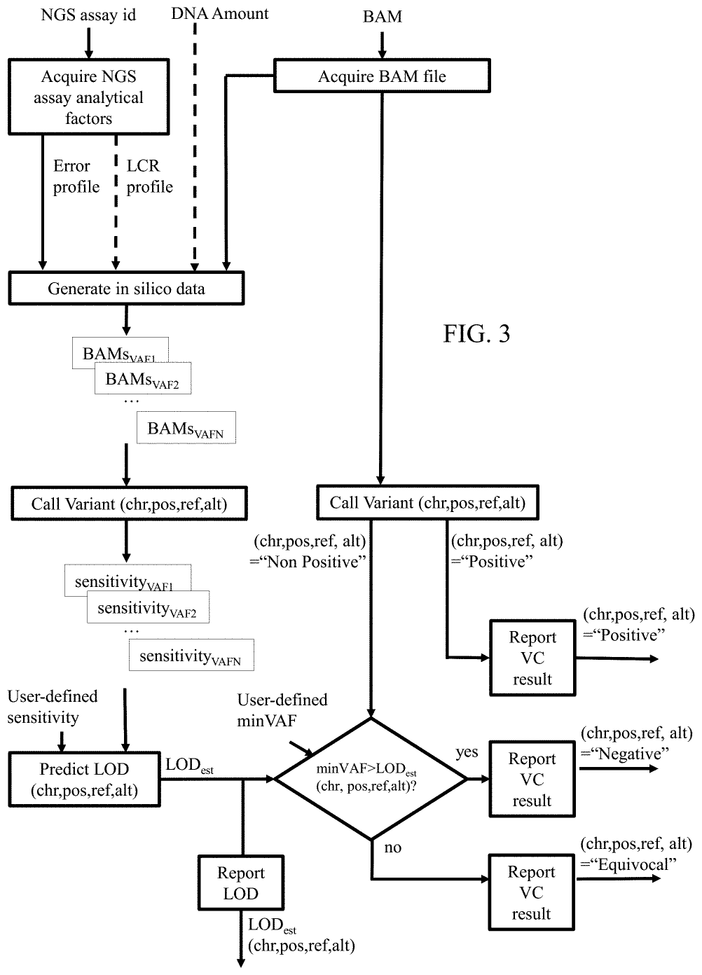

FIG. 3 illustrates a possible workflow for the proposed LOD-aware variant caller 200 to estimate a limitation LODₑₛₜ of detecting a nucleic acid sequence variant (chr,pos,alt,ref) in the alignment data (for instance a BAM file, but other embodiments are also possible) generated by a next-generation-sequencing assay from a patient sample, and for reporting the variant calling status as positive, negative or equivocal. Preferably, the variant may be an SNV or an INDEL, but other embodiments are also possible.

The proposed LOD-aware variant caller 200 may first acquire a BAM file for the patient sample, and determine, with a conventional variant calling module 123, the status of variant (chr,pos,alt,ref) in the alignment data.

If the variant call is positive, the LOD-aware variant caller 200 may then simply report its status as “positive” for instance in a VCF file. Yet if the variant call is non-positive, instead of either ignoring it or reporting it as negative (even though it is possibly a false negative), the LOD-aware variant caller 200 may apply the following steps:
Obtain a molecular count measurement for the patient sample at the genomic position (chr,pos) of said variant;
Obtain one or more analytical factors of the NGS assay used to process the patient sample, such as an NGS assay error profile and optionally an LCR profile;
Produce, with a statistical model synthetic alignment data for one or more simulated VAFs as a function of the measured molecular count and of the analytical factors of the NGS assay; and
Estimate, from the synthetic alignment data, the detection sensitivity of said variant caller as a function of one or more of the simulated VAFs, for said assay, said DNA sample and said variant (chr, pos, ref, alt).

In a possible embodiment, the LOD-aware variant caller generates, with a statistical model such as for instance a generative model, in silico alignment data as a function of the measured coverage, the measured molecular count and the analytical factors of the NGS assay. To produce in silico data, a number of simulated VAFs values may be selected in a range such as for example from 0.5%, 0.1%, 0.01% or less to 30%, 40%, 50% or more, depending on the application. For each of these simulated VAFs, a synthetic alignment data set may be produced as a BAM file, or alternately as a set of NGS data alignment features, as is suitable for processing by the conventional variant caller module 123. The conventional variant caller module 123 may then be called on each synthetic alignment data set to estimate the detection sensitivity specifically at the simulated VAF used to generate this data set. The LOD-aware variant caller may accordingly estimate a detection sensitivity function which is specific to the assay, the sample and the variant, by combining the sensitivity predictions of the different simulated datasets (each with a different simulated VAF).

In a further possible embodiment, the LOD-aware variant caller 200 may acquire a user-defined minimal variant allele fraction of interest (mVAF) for variant (chr, pos, ref, alt), and classify the variant status as negative or equivocal as a function of the estimated detection sensitivity function and mVAF for this variant. To this end, the LOD-aware variant caller 200 may obtain a user-defined desired sensitivity for calling said variant as positive, estimate, from the estimated detection sensitivity function, the sensitivity for said mVAF, and classify the variant status as negative or equivocal as a function of said desired and estimated sensitivity, but other embodiments are also possible.

The LOD-aware variant caller 200 may accordingly report the variant calling status and/or the estimated sensitivity to the end user for the called variant (chr,post,ref,alt), for instance in a file format extension to the VCF format for variant reporting, processing and/or storage, and/or using a dedicated graphical user interface to directly display the results to the end user.

In another possible further embodiment, the LOD-aware variant caller 200 may obtain a user-defined target sensitivity for variant (chr, pos, ref, alt) and estimate, from the estimated detection sensitivity function, a limit of detection LODₑₛₜ value for this variant as the lowest VAF detectable with a sensitivity larger or equal to the user-defined sensitivity.

The LOD-aware variant caller 200 may also obtain both a user-defined minimal variant allele fraction of interest (mVAF) and a user-defined desired sensitivity for calling variant (chr, pos, ref, alt) as positive, estimate from the estimated detection sensitivity function a limit of detection LODₑₛₜ value as the lowest simulated VAF detectable with a sensitivity larger or equal to the user-defined sensitivity and classify the variant status as negative if the mVAF for said variant is larger or equal to LODₑₛₜ, or as equivocal otherwise.

The LOD-aware variant caller 200 may accordingly report the estimated LODₑₛₜ value and/or the variant calling status result to the end user for the called variant (chr,post,ref,alt), for instance in a file format extension to the VCF format for variant reporting, processing and/or storage, and/or using a dedicated graphical user interface to directly display the results to the end user.

### Experimental Results

A non-limiting exemplary embodiment will now be described, based on a carefully controlled experiment to define the factors governing NGS assay limitations, using circulating tumor DNA profiling as a paradigm. This uncovered a complex interplay between technical factors and genomic context, enabling the development of the in-silico method that predicts the limit of detection (LOD, the lowest detectable variant fraction) for every position interrogated by an assay. This method may be integrated into an LOD-aware variant calling framework 200 that uses LOD predictions to flag potential false negatives. Applying this to 580 clinical samples predicted all but one false negative call, resulting in an inter-assay concordance of 99%. The proposed approach improves the reliability, reproducibility and transparency of genetic testing, with widespread benefits for clinical trials, technology development and patient care.

The proposed approach may include an LOD-aware variant calling framework 200 that predicts and reports LODs alongside NGS results. This approach may be developed to operate with a diversity of NGS assays. For the carefully controlled experiment, ctDNA assays were used, as these represent a challenging test case that can benefit greatly from reliability improvements.

A complex interplay between technical factors and genomic position defines LOD, with contributing factors including input DNA amount, library conversion rate, sequencing coverage and PCR/sequencing error rate. Variation in these factors leads to a remarkable variability in LOD between samples, NGS technologies and genomic positions. Drawing upon these insights, an in-silico approach may be developed to accurately predict the LOD for every genomic position interrogated by an NGS assay. It may then be integrated into an LOD-aware variant calling framework 200 that introduces a third label for variant calls based on technical limitations. Specifically, negative calls may be triaged into high confidence results versus potential false negatives (now labeled “equivocal”).

### Example Experiments

A1 (NGS assay A1, amplicon-based); A2 (NGS assay A2, capture-based); A3 (NGS assay A3, capture-based with molecular barcoding); BEAMing (Beads, Emulsion, Amplification and Magnetics); cfDNA (cell-free DNA); ctDNA (circulating tumor DNA); dPCR (digital PCR); ddPCR (droplet digital PCR); EGFR (gene coding epidermal growth factor receptor); gDNA (genomic DNA); KRAS (gene coding K-Ras protein); LCR (library conversion rate); LOD (limit of detection); NGS (next-generation sequencing); NSCLC (non-small cell lung cancer); rsctDNA (reference standard circulating tumor DNA); SNVs (single nucleotide variants); VAFs (variant allele frequencies or fractions).

For the purposes of this disclosure, the following examples should be interpreted as non-limiting.

### Example 1: LOD is Determined at Multiple Scales

The limit of detection (LOD) of next-generation sequencing (NGS) assays was defined as described below.

### Materials and Methods

Clinical cfDNA samples: Samples were collected within the framework of the CIRCAN (“CIRculating CANcer”) study, which is a routine program established to comprehensively evaluate tumor biomarkers in cell-free DNA (cfDNA) from non-small cell lung cancer (NSCLC) patients at the Lyon University Hospital (Hospices Civils de Lyon, HCL). The main inclusion criteria were (i) that patients were histologically or cytologically diagnosed as having metastatic NSCLC, and (ii) that these patients had undergone molecular testing for epidermal growth factor receptor (EGFR) mutations in tumor biopsies (routinely performed in France) (Garcia et al., 2017, Oncotarget, Vol 8, No 50; Garcia et al., 2018, Oncotarget, Vol 9, No 30). Patient inclusion was initially limited to patients treated in the HCL and in countryside hospitals in the Rhone-Alpes region.

Plasma was prepared from 10-25 mL of blood collected in K2 EDTA (ethylenediaminetetraacetic acid) tubes (BD, 367525, 18 mg). All blood samples were delivered to the laboratory within 24 hours of collection. Detailed pre-analytical considerations have previously been published (Garcia et al., 2017, supra). CfDNA was extracted from 4 mL or 8 mL of plasma using the QIAamp Circulating Nucleic Acid Kit (Qiagen, Cat No 55114, Valencia, Calif., USA), with a Qiagen vacuum manifold following the manufacturer's instructions. CfDNA was then eluted in a final volume of 60μL of elution buffer (AVE; Qiagen, part of Cat No 55114) depending on the volume of plasma used for the extraction (3 mL or 8 mL).

Preparation of Reference ctDNA: (1) rsctDNA (Reference Standard Circulating Tumor DNA) and (2) Sonicated gDNA (Genomic DNA)

Enzymatic shearing (rsctDNA): Nucleosomal DNA was generated from seven different human cell lines (GM07048, GM14638, GM14097, GM14093, GM12707, GM12815 and GM11993, Coriell Institute, with the EZ Nucleosomal DNA Prep (Zymo Research, Cat. No. D5220) using the Atlantis dsDNase treatment (Zymo Research, part of Cat. No. D5220) according to the manufacturer's instructions with minor modifications, as follows. After collection, cells were stored at −80° C. For each cell line, a pellet of 10⁶ cells was thawed on ice, resuspended in 1 ml ice-cold lysis buffer (10 mM Tris-HCl pH 7.5, 10 mM NaCl, 3 mM MgCl₂, 0.5% NP-40, 0.5 mM Spermidine) and incubated for 5 min on ice. The resulting nuclei were then washed once with 200 μl ice-cold Atlantis digestion buffer, then resuspended in 100 μl Atlantis digestion buffer with 0.25 U Atlantis dsDNAse (double-stranded DNA-specific endonuclease) and incubated for 1 h at 42° C. To stop the digestion, 18 μl of Stop solution (75 mM EDTA, 1.5% SDS, 0.7 M NaCl) was added. Samples were then treated with RNaseA (ribonuclease) (0.3 mg/μ1) for 15 min at 42° C., then with proteinase K (1 mg/ml) for 30 min at 37° C. Following enzymatic treatments, DNA was column purified with the Zymo Genomic DNA Clean & Concentrator kit (Zymo Research, Cat. No. D4065). To remove large and small DNA fragments, samples were subjected to a dual size selection using AMPure XP Beads (Beckman Coulter) with bead:DNA ratios of 0.7× and 1.5×. After fluorometric quantification, the nucleosomal DNAs of GM07048, GM14638, GM14097, GM14093, GM12707 and GM12815 were spiked into the nucleosomal DNA of GM11993, generating DNA mixes (D1, D2 and D3; dilution 1, 2 and 3 respectively) with variants at the indicated variant fractions (VAF):

| | | Expected | Expected | Expected |
|---|---|---|---|---|
| Variant | Gene | VAF D1 | VAF D2 | VAF D3 |
| 1:115256414_C:T | NRAS | 0.50% | 0.25% | 0.10% |
| 2:29445458_G:T | ALK | 0.50% | 0.25% | 0.10% |
| 2:198266862_A:T | SF3B1 | 0.50% | 0.25% | 0.10% |
| 2:209113192_G:A | IDH1 | 0.50% | 0.25% | 0.10% |
| 2:212566786_G:T | ERBB4 | 0.50% | 0.25% | 0.10% |
| 3:138665029_G:C | FOXL2 | 0.50% | 0.25% | 0.10% |
| 3:138665064_G:A | FOXL2 | 0.50% | 0.25% | 0.10% |
| 4:1803704_T:C | FGFR3 | 1.00% | 0.50% | 0.20% |
| 4:55152040_C:T | PDGFRA | 1.50% | 0.75% | 0.30% |
| 4:55593464_A:C | KIT | 0.50% | 0.25% | 0.10% |
| 4:55602765_G:C | KIT | 0.50% | 0.25% | 0.10% |
| 7:55249063_G:A | EGFR | 1.00% | 0.50% | 0.20% |
| 7:55259450_C:T | EGFR | 0.50% | 0.25% | 0.10% |
| 7:116339282_G:A | MET | 0.50% | 0.25% | 0.10% |
| 7:116339672_C:T | MET | 0.50% | 0.25% | 0.10% |
| 7:116411923_C:T | MET | 0.50% | 0.25% | 0.10% |
| 9:21970916_C:T | CDKN2A | 0.50% | 0.25% | 0.10% |
| 10:43610119_G:A | RET | 0.50% | 0.25% | 0.10% |
| 10:43613843_G:T | RET | 4.00% | 2.00% | 0.80% |
| 10:43615633_C:G | RET | 0.50% | 0.25% | 0.10% |
| 11:534332_G:A | HRAS | 0.50% | 0.25% | 0.10% |
| 12:112888239_C:T | PTPN11 | 0.50% | 0.25% | 0.10% |
| 17:7576841_A:G | TP53 | 0.50% | 0.25% | 0.10% |
| 17:7579579_C:T | TP53 | 0.50% | 0.25% | 0.10% |

Mechanical shearing (sonicated gDNA): Sonicated genomic DNA was prepared using an E220 Evolution sonicator (Covaris) according to the manufacturer's instructions, to obtain an average size of 150 bp. Briefly, for each sample, 1 μg of DNA in 55 μl of TE buffer was added to a Rack E220e 8 microTUBE Strip V2 and sheared using the following parameters: Peak Incident Power (W): 75, Duty Factor: 15%, Cycles per Burst: 500, Treatment Time: 360 s.

Fragment size analysis of reference ctDNA and ctDNA from clinical samples: The cfDNA size from clinical samples were assessed using the Agilent 2100 BioAnalyser (Agilent Technologies, Santa Clara, Calif., USA) and the DNA High Sensitivity kit (Agilent Technologies, Santa Clara, Calif., USA, 5067-4626 & 5067-4627). Two size-standardized internal controls (of 35 bp and 10,380 bp) and a DNA ladder (15 peaks) were used in each bioanalyser runs. The profile of fragment sizes was generated using the 2100 Expert Software (Agilent Technologies, Santa Clara, Calif., USA).

### NGS Library Preparation and Sequencing

NGS assay A1. Targeted libraries were created using a multiple targeted amplicon kit (Accel-Amplicon 56G Oncology Panel v2, AL-56248, Swift Biosciences) according to the manufacturer's instructions. The kit enables the detection of mutations present in a set of clinically relevant genes implicated in cancers. cfDNA samples with inputs varying from 2.2 to 37.7 ng were subjected to an initial multiplex PCR using the panel specific set of primers and 25 cycles of amplification. PCR products were purified using AMPure beads (Beckman Coulter). Finally, dual-index adapters, provided as part of the kit, were ligated to the purified PCR products prior to a final AMPure bead purification step (Beckman Coulter).

NGS assay A2: Targeted libraries were created using capture-based enrichment technology. First, 10-50 ng of input cfDNA was end-repaired and A-tailed, followed by ligation to Illumina dual-indexed adapters. Ligation products were purified using AMPure beads (Beckman Coulter) and further amplified by PCR for 10 to 14 cycles depending on the amount of input DNA. Amplified libraries were cleaned-up using AMPure beads (Beckman Coulter) and then libraries pooled to give a total of 1.8 μg. The pools were mixed with human Cot-1 DNA (Life Technologies) and xGen Universal Blockers-TS Mix oligos (Integrated DNA Technologies) and lyophilized. Pellets were resuspended in a hybridization mixture, denatured for 10 min at 95° C. and incubated for 4-16 h at 65° C. in the presence of biotinylated probes (xGEN Lockdown IDT®). The probe panel spanned 170 Kb and covers a set of clinically relevant genes implicated in cancer that partially overlaps with the panel of assay A1. Probe-hybridized library fragments were captured with Dynabeads M270 Streptavidin (Invitrogen) and then washed. The captured libraries were amplified by PCR for 14 cycles and cleaned-up using AMPure beads (Beckman Coulter).

NGS assay A3. Targeted libraries were created using a capture-based enrichment technology including molecular barcodes. First, 10-50 ng of input cfDNA was end-repaired and A-tailed, followed by ligation to short y-shaped adapters with a double-stranded molecular barcode of 4-5 bp. The ligation products were purified with AMPure beads (Beckman Coulter) and then amplified for 10 to 14 cycles (depending on the amount of input DNA) using Illumina-compatible primers with dual-indices. Amplified libraries were cleaned-up with AMPure beads (Beckman Coulter). Targeted enrichment of pooled libraries was performed as for NGS assay A2 but using one more PCR cycle (i.e. 15 instead of 14). This is because the probe panel for assay A3 has a smaller footprint (56 Kb) than that of assay A2 (note that panel A3 also targets genes clinically relevant for cancer). PCR products were finally purified using AMPure beads (Beckman Coulter). Panel A3 targets genes clinically relevant for cancer which partially overlap with the targets of assays A1 and A2.

### Sequencing

Prior to sequencing, libraries were quantified by qPCR and normalized to 0.5 nM for NGS assay A1 and 4 nM for NGS assays A2 and A3. All libraries were sequenced using a NextSeq 500 sequencer (Illumina) with either a Mid-Output or High-Output kit as specified by the manufacturer. Paired-end sequencing reads of 150 bases were generated (2×150 cycles) together with two index reads of 8 bases. Sonicated gDNA and rsctDNA libraries were loaded on the sequencer such that coverage was proportional to the input material and comparable between the different size panels (A1: 2800×/ng; A2: 2200×/ng, and 1900×/ng, corresponding to at least 6 reads per molecule on average). This guarantees that most molecules converted into library were sequenced at least once. Clinical samples were sequenced as per the assay manufacturers' instructions.

### Data Processing and Standard Variant Calling

NGS data demultiplexing, pre-processing and alignment: Demultiplexing (and molecular barcode trimming for assay A3) was performed on base-call files (BCL) using bcl2fastq2 and the resulting FASTQ files aligned against the human genome reference Hg19 (GRCh37.p5) using bwa (Li & Durbin, 2009, Bioinformatics 25, 1754-1760). For each genomic position a pileup was performed by counting reads with a Phred quality score >15 supporting the reference or alternative allele. For assay A3, groups of PCR duplicates were collapsed to consensus sequences prior to the pileup step (Schmitt et al., 2012, PNAS 109, 14508 LP-14513). First, pairs of reads were grouped into DNA molecules using their mapping positions and molecular barcode sequences. Molecules for which only one of the two strands were sequenced were discarded. For each remaining molecule a base call consensus was obtained for each strand. 70% base call consistency within a strand to form a consensus was required. Finally, a consensus was obtained for the two strands, producing a single consensus sequence for each DNA molecule. Alternative bases needed to be present in both single strand consensuses to be retained in the final consensus.

Standard variant calling: First, a background noise model was generated for each assay, genomic position and mutation (three SNVs, insertion and deletion) using NGS data from characterized samples. For assay A1, 136 Tru-Q samples (Tru-Q 0, 5, 6 and 7, Horizon Discovery) routinely included as controls in clinical runs were used. For assay A2, 181 germline blood samples included in clinical runs were used. For assay A3, 10 negative samples included as controls with the rsctDNA samples were used (these are samples produced entirely from the background cell line). For a given depth and mutation (three single nucleotide variants (SNVs), insertion or deletion) the background noise model is a fitted distribution of the number of alternative reads produced by technical noise, such as sequencing errors. For assays A1 and A2 a beta-binomial distribution to the measured background noise profile was fitted, which was able to represent the observed overdispersion (Ramu et al., 2013, Nat. Methods 10, 985-987). For assay A3 technical noise was lower as PCR duplicates were collapsed to consensus sequences. This permitted fitting of a simpler Binomial model. Using flat priors, the beta-binomial was fitted to the data using maximum likelihood estimation, while the mean a posteriori approach was used to obtain the rate for the binomial model. For model fitting, confirmed variants were excluded (as these would skew the background noise estimation and were not noise). Also excluded were positions with a VAF larger than 25%, as these were likely to represent true variants. In cases where a position had a variant in all samples and thus no data were available for the fit, the parameters were set to the average of the background noise of neighboring positions. To call variants, the probability that the observed number of reads supporting the alternative allele N_{ALT} was generated by the background noise model, was calculated i.e.,

P(N_{ALT}^{Noise}>N_{ALT}|N,θ) (Eq. 1)

where θ represents the parameters of the fitted beta-binomial or binomial for a given genomic position and variant type (three SNVs, insertion or deletion). The negative logarithm of this probability was taken, and variants were called based on whether this score was above or below a defined threshold. The threshold value a was chosen to ensure a similar false positive rate, measured across all reference positions for the reference standard samples, and was 50, 50 and 7 for assays A1, A2 and A3 respectively.

### Collision Rate Calculation

As used herein, the term “collision” means the event in which two, or more than two, different DNA molecules were fragmented the exact same way, and thus cannot be distinguished by their mapping position alone. The collision rate is the rate at which such events occur, and a high collision rate indicates that molecular barcodes are necessary to accurately quantify molecular counts. More precisely, the number of mapping positions at which more than one molecule aligned was counted and divided by the total number of mapping positions occupied in the data.

### Theoretical LOD for a Given Input Amount

To evaluate the impact of input material on sensitivity a minimal model was developed that assumes that coverage is in excess (i.e. all molecules incorporated into the library will be sequenced) and neglects sequencing noise. Thus, the ability to detect variants is solely limited by the number of molecules in the input material supporting the variant, M_{ALT}, and the library conversion rate, LCR. Here it was assumed that M_{ALT} follows a Poisson distribution with mean 300×Input×VAF×LCR, and that the probability of detecting a variant with a given VAF and input material was given by:

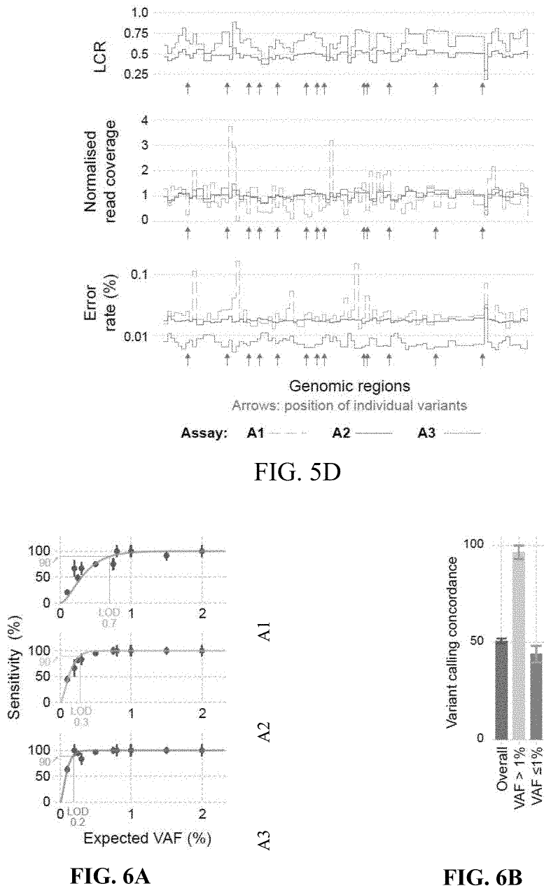

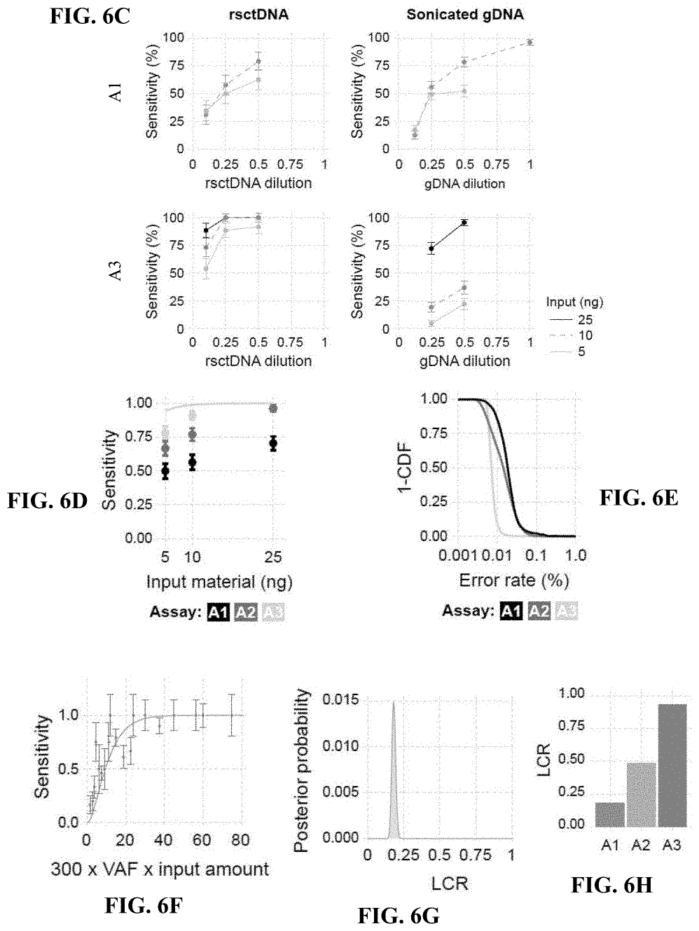

P(M_{ALT}>k|VAF,Input,LCR) (Eq. 2)

where k is the minimum number of molecules required to make a call. It was assumed that at least two molecules were required to make call. An LCR of 100% for the maximum theoretical sensitivity was used. These data are presented in FIG. 6D. As this figure aggregates different VAFs, the average sensitivity over the VAFs present in the analyzed samples was computed. This model was also used as a basis to infer the LCR for assay A3 (Library conversion rate estimation, NGS assay A3 below) (FIG. 6F-H).

### Library Conversion Rate Estimation

The library conversion rate was defined as the fraction of DNA molecules present in the sequencing data divided by the number of DNA molecules present in the sample, given by the number of haploid genomes per ng (300) multiplied by the mass of input material (in ng). For this calculation, the number of molecules in the sample needs to be determined.

NGS assay A2 and A3: For assay A3 the number of molecules can readily be measured by identifying PCR duplicates using the fragments' mapping positions and molecular barcodes. The LCR was computed per targeted region by retaining fragments that overlapped the center of the region, and an average LCR computed by averaging over genomic regions. The same method was applied for assay A2, but since this assay was not equipped with molecular barcodes the resulting LCR was likely to be underestimated, as two molecules mapping as the same position (a collision) was counted as one. However, replicates were used to estimate the collision rate and correct for this bias. The frequency at which molecules map at the same position can be estimated by counting the number of mapping positions occupied in both replicates and dividing by the total number of occupied mapping positions. It was found that for 5 ng of input material the collision rate was 6%. The LCR was thus corrected by inflating the observed number of molecules by the collision rate.

NGS Assay A1: Since assay A1 was not equipped with molecular barcodes and the mapping positions were the same for all fragments in an amplicon, it was not possible to rely on molecular counts to estimate the LCR for assay A1. Therefore, the observed variant calls for rsctDNA samples exploited instead. The model for the number of molecules supporting the variant, M_{ALT}, defined as above (in Theoretical LOD for a given input amount) was used, and it was assumed that at least two molecules were required to make a call, as it gave good agreement with the data (FIG. 6A). For a given variant i with a VAF VAFᵢ and input amount Inputᵢ the probability of the call (i.e. called or missed) was given by:

$$( Eq . 3 )$$

$$P ( C a l {l}_{i} \vert L C R ) = \{ \begin{matrix}P ( {M}_{A L T} > k \vert V A {F}_{i} , {Input}_{i} , LCR ) if the variant was called \\ P ( {M}_{A L T} \leq k \vert V A {F}_{i} , {Input}_{i} , LCR ) if the variant was missed\end{matrix}$$

The likelihood of observing the calls, as a function of LCR, was then given by:

$$P ( Calls \vert LCR ) = {\prod }_{i = 1}^{N} P ( C a l {l}_{i} \vert L C R ) \qquad ( Eq . 4 )$$

Assuming a flat prior for the LCR, the likelihood was then numerically integrated to obtain the posterior distribution P(LCR|Calls), which is plotted in FIG. 6B.

Variant concordance analysis: To measure the concordance between the three NGS assays, or between NGS assays and PCR-based methods, the fraction of confirmed variants that had the same call status (positive or negative) in all assays was computed. For LOD-aware variant-calling all variants that had at least two non-equivocal calls were selected, and concordance computed amongst these. For example, the situation in which two assays made a positive call and the third one made an equivocal call was counted as concordant, while the situation in which only one of the three assays made a non-equivocal call was excluded from the concordance analysis.

### Results

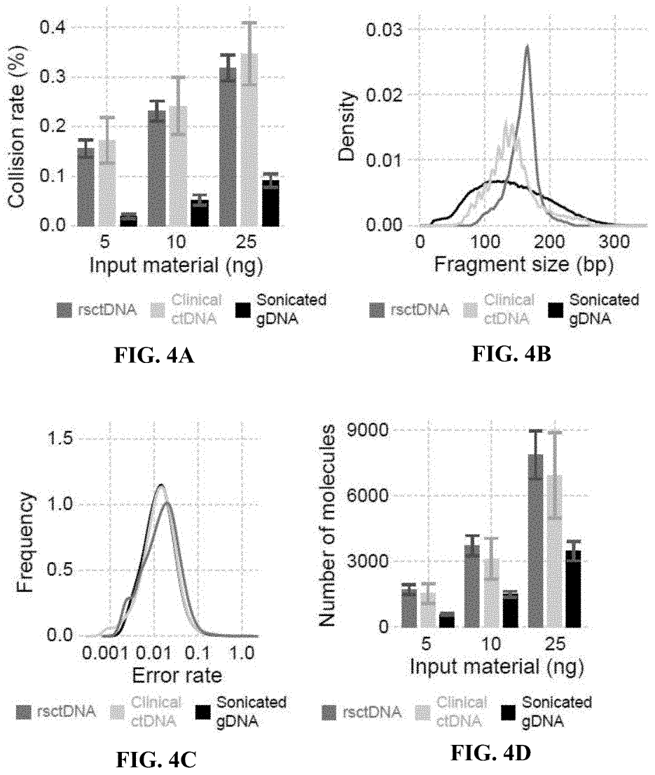

To accurately predict the LOD of NGS assays required a detailed understanding of how LOD is defined. Therefore, there was a need for an experimental system where technical factors such as input amount, assay biases and sequencing errors could be studied individually and in combination. This may be impractical using clinical samples, due to their heterogeneity and scarcity, so an artificial reference material may be sought. A range of these were available, and as it was unclear which was most commutable with clinical samples (Geeurickx & Hendrix, 2020, Mol. Aspects Med. 72, 100828) two possibilities were tested. The first, “reference standard ctDNA” (rsctDNA), comprises DNA purified from endonuclease-digested chromatin and mimics circulating tumor DNA (ctDNA) production in vivo (Snyder et al., 2016, Cell 164, 57-68). Commutability of sonicated gDNA and rsctDNA, from endonuclease-digested chromatin with clinical ctDNA, was assessed using a capture-based NGS assay with molecular barcodes (assay A3; FIG. 4). Collision rate (FIG. 4A), fragment size distribution (FIG. 4B), distribution of error rates at genomic positions (n=6400) within the target region shared by all assays (FIG. 4C), and the average number of unique molecules covering each interrogated genomic position (FIG. 4D) were assessed.

Comparing NGS libraries from rsctDNA and clinical ctDNA revealed similar molecular features (Mouliere et al., 2018, Sci. Transl. Med. 10, eaat4921), whereas the second reference material, sonicated genomic DNA (gDNA), produced a less complex library (FIG. 4A-D). It was concluded that rsctDNA is the more clinically analogous reference.

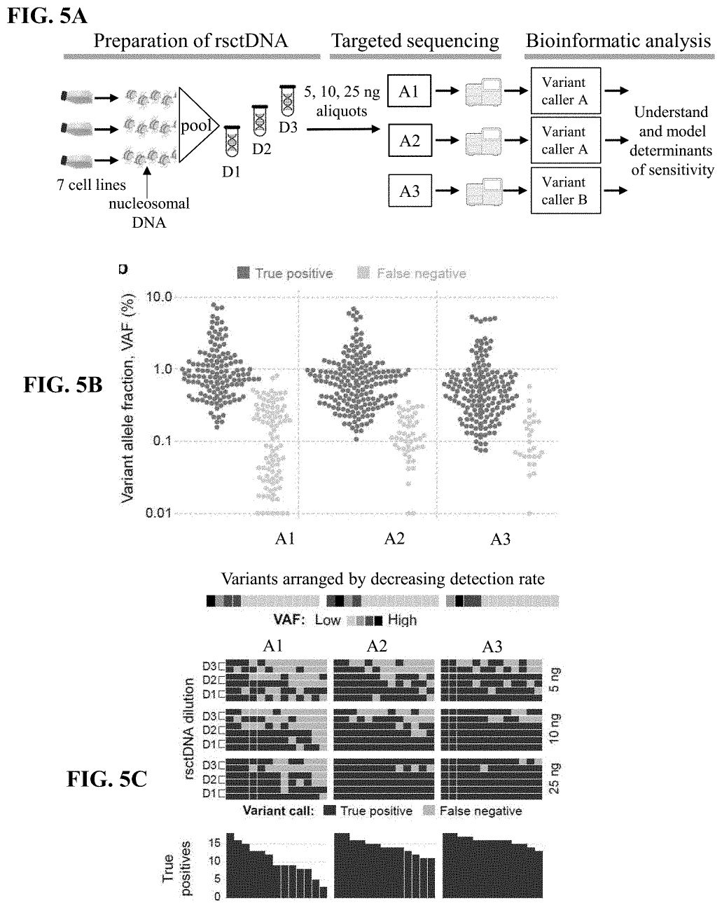

Next, an NGS experiment using rsctDNA to examine determinants of LOD was designed, as shown in FIG. 5A. Three dilutions of rsctDNA (i.e., D1, D2 and D3), each containing 13 variants at various VAFs (i.e., D1 0.5-4%; D2 0.25-2%; D3 0.1-0.8%), were prepared by mixing DNA isolated from endonuclease-digested chromatin extracts (from seven cell lines). Three rsctDNA amounts (i.e., 5 ng, 10 ng and 25 ng), three dilutions (i.e., D1, D2 and D3, each containing 13 single nucleotide variants, SNVs, with a clinically relevant VAF range of 0.1-4% (Matsumoto et al., 2020, Lung Cancer 139, 80-88; Jiang et al., 2019, Mol Med Rep 20, 593-603)), and three NGS technologies (amplicon-based (assay A1), capture-based (assay A2) and capture-based with molecular barcoding (assay A3)) were included. NGS data were used for variant calling, and the results used to assess concordance between NGS assays and determine factors governing sensitivity/LOD. It was first evaluated whether this setup yielded a similar analytical performance to that observed in clinical studies, by examining variant calls for the known SNVs. This revealed abundant false negatives, particularly for low VAFs (FIG. 5B), as seen in clinical studies. Consequently, the overall LOD was 0.2-0.7% and concordance between assays low (44% for VAFs □1%) (FIG. 6A-B). Notably, the sensitivity (proportion of confirmed SNVs detected) of assay A3 was lower when this experiment was repeated using sonicated gDNA (FIG. 6C), confirming that rsctDNA is the better reference choice. Overall, the low inter-assay concordance resembles that seen in clinical studies (22-55%) (Stetson et al., 2019, supra; Kuderer et al., 2017, supra; Jovelet et al., 2016, Clin. Cancer Res. 22, 2960 LP-2968), confirming that this model was realistic.

To determine which factors influence LOD, variant calls (true positive or false negative) were plotted for the 13 SNVs arranged by rsctDNA dilution, NGS assay, and input amount (FIG. 5C). The manner in which sensitivity, i.e. the proportion of true positives, varies between these conditions was then examined. As sensitivity is inversely related to LOD, this reveals factors contributing to LOD. Thus, sensitivity may be lowest for low input amounts or VAFs (FIG. 5C, FIGS. 6A and D). There were also clear differences between NGS technologies, which could be due to variation in the extent to which PCR or sequencing noise obscure faint signals. Indeed, the most sensitive assay (A3) had the lowest error rate (FIG. 6D-E). Nonetheless, assay A3 did not achieve the maximum sensitivity possible given the amount of input material (FIG. 4D), implying that additional factors also limit sensitivity. One candidate was library conversion rate (LCR), the efficiency with which DNA is incorporated into an NGS library. Therefore, the LCR was computed for each assay, either by counting detected molecules (distinct fragments in the NGS library) or, as this approach was not possible for assay A1, using a model that assumes LCR is limiting (FIG. 6F-H). Overall, LCR correlated with sensitivity. It was therefore concluded that LCR, error rate, and input amount may all govern sensitivity and thus LOD.

Importantly, for a given assay, input amount, and rsctDNA dilution, variants with the same VAF were detected with markedly different sensitivities (FIG. 5C, light grey (low VAF) variants). This implies that LOD depends strongly on genomic context. Consistent with this, determinants of LOD (error rate, sequencing coverage and LCR) fluctuate between genomic positions (FIG. 5D). It was concluded that to accurately predict LOD, the complex interplay between technical factors acting at assay, sample and nucleotides levels must be taken into account.

### Example 2: Adaptive LOD Predictions Resolve NGS Assay Discordance

A variant calling framework that identifies and reports potential miscalls was developed as follows.

### Materials and Methods

Materials and methods as in Example 1, in addition to the following:

LOD-Aware Variant Calling

Generative model: To predict LOD and sensitivity more generally than described in Example 1 (i.e. for different amounts of input material or higher VAFs), and to take account of background noise, a generative model to simulate the entire NGS workflow was developed, with simulated data then interpreted by a variant caller.

The simulated data may contain all features normally required by whichever variant caller is used (read depth, count of reference and alternative nucleotides at a given position, base quality score, etc.) and be supplied in a format that the variant caller accepts. In the case of our own variant caller, this information was supplied directly as numerical inputs, whereas for VarDict (see below Example 4) the information must have been supplied in the form of synthetic bam files. Note that if the variant caller requires pre-processing of reads, such as collapsing reads from the same DNA molecule (“read groups” or “read families”) to create consensuses, this must be reflected in the simulated data (i.e., if the variant caller requires collapsed consensus sequences as an input, the simulated input data should correspond to consensus sequences rather than individual sequencing reads). The simulated data should follow the guidelines of the variant caller developer—for example, if the developer recommends using consensus sequences from read groups with at least two read pairs, the LOD-aware approach should be calibrated using such datasets. This will ensure that the properties of the simulated data, such as error rate, mimic the real data and are appropriate for the variant caller being used.

Variant caller: A variant caller can be seen as a function VC mapping a set of observed features F (e.g., like coverage, number of reads supporting the variants, and total number of reads at each genomic position) to a score that is used to take a decision, given a threshold a, on the presence of a variant: VC(F)>α. To obtain the probability distribution of the features F (e.g., reference and alternative read counts) given a predefined set of parameters (e.g., Input material, VAF, LCR, error rate and sequencing read coverage), i.e., P(F|θ), a generative model was used (Generative model for NGS assay A1, A2 and A3, below). The predicted sensitivity could then be computed by integrating the positive calls over the set of features:

Predicted sensitivity=∫_{F}(VC(F)>α)P(F|θ) (Eq. 5)

Depending on the complexity of the feature set and of the generative model this integral could either be solved analytically, integrated numerically, or approximated using sampling techniques. Here, since our feature set was small and our generative model was relatively simple, numerical integration was used to solve the integral. Importantly, since the predicted sensitivity is a function of the VAF (one of the features within the feature set F), one could solve the equation above for the VAF corresponding to a desired predicted sensitivity (i.e. the LOD).

Generative model for NGS assay A1 and A2: The generative model for assays A1 and A2 aimed at producing the distribution of the number of reads supporting the variant, N_{ALT}, given the total number of reads N (coverage depth, known from the NGS data), the input material, the LCR, a VAF and a background noise model. It was reasoned that at low variant fraction, the fluctuations in the number of alternative molecules and alternative reads would play a larger role than fluctuations in the total number of molecules and reads. Therefore, integration was performed over the distributions for the former and averages used for the latter. Thus, the mean total number of molecules M was on average 300×Input×LCR, and the number of molecules supporting the variant, M_{ALT}, followed a Binomial distribution with mean 300×Input×LCR×VAF. It was assumed that molecules were amplified homogeneously into pools of PCR duplicates and reads were sequenced randomly from these pools. Thus, the mean number of sequenced reads per molecule was given by λ=N/M, and the total number of reads supporting the variant N_{ALT} follows a Poisson distribution with mean λ×M_{ALT}. The contribution of the background noise was added to this, with the number of artefactual reads supporting the variant following a beta-binomial distribution with parameters estimated as explained above (Standard variant calling). The predicted sensitivity was finally computed as:

$${\sum }_{{M}_{A L T}} {\sum }_{{N}_{A L T}} {\sum }_{{N}_{A L T}^{N o i s e}} ( V C ( {N}_{A L T} + {N}_{A L T}^{N o i s e} , N ) > α ) P ( {N}_{A L T}^{N o i s e} ) P ( {N}_{A L T} \vert {M}_{A L T} ) P ( {M}_{A L T} ) \qquad ( Eq . 6 )$$

Instead of integrating on the whole support of the distributions, the sum was restricted to the 10⁻⁵ and 1-10⁻⁵ quantiles of the distributions, which was sufficient to produce an accurate estimation of predicted sensitivity.

Generative model for NGS assay A3: Since data produced by assay A3 were deduplicated to form consensuses, each resulting read-pair corresponds to a single DNA molecule, so the generative model for A3 only needed to produce the distribution of the number of alternative molecules supporting the variant M_{ALT} given the total number of molecules M (coverage of duplexes, known from the NGS data), a VAF and a background noise model. As in the previous section, the number of alternative molecules followed a Binomial distribution with mean M×VAF, and the contribution of the background noise (Binomial distribution) was added to obtain the total number of alternative molecules provided to the variant caller. Thus, the predicted sensitivity was:

$${\sum }_{{M}_{A L T}} {\sum }_{{N}_{A L T}^{N o i s e}} ( V C ( {M}_{A L T} + {M}_{A L T}^{N o i s e} , M ) > α ) P ( {M}_{A L T}^{N o i s e} ) P ( {M}_{A L T} ) \qquad Eq . ( 7 )$$

### Results

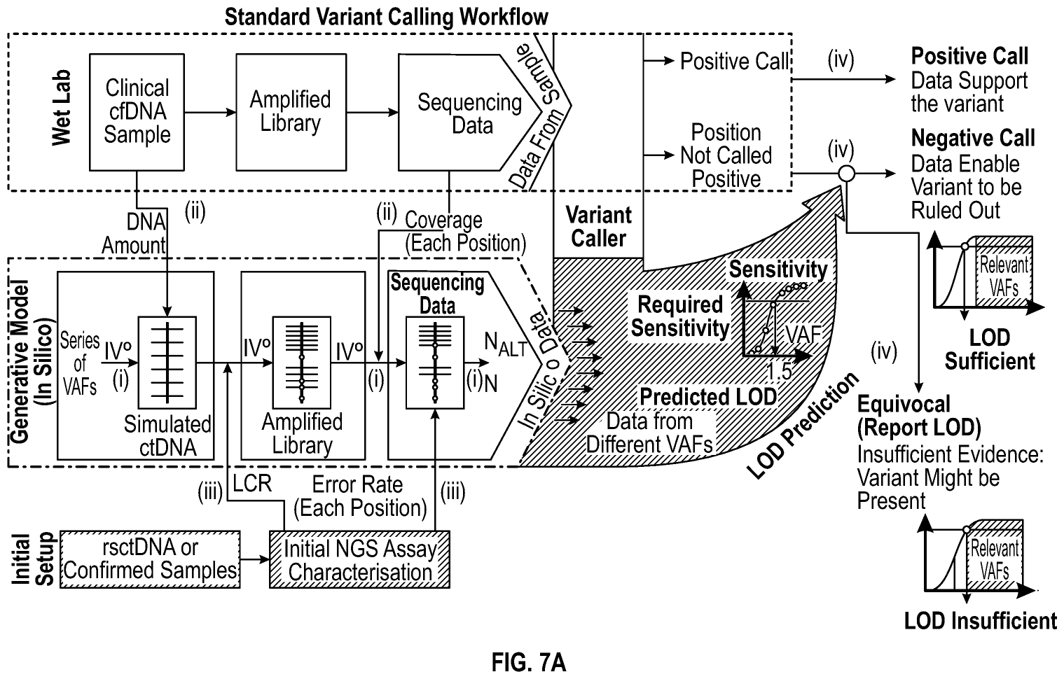

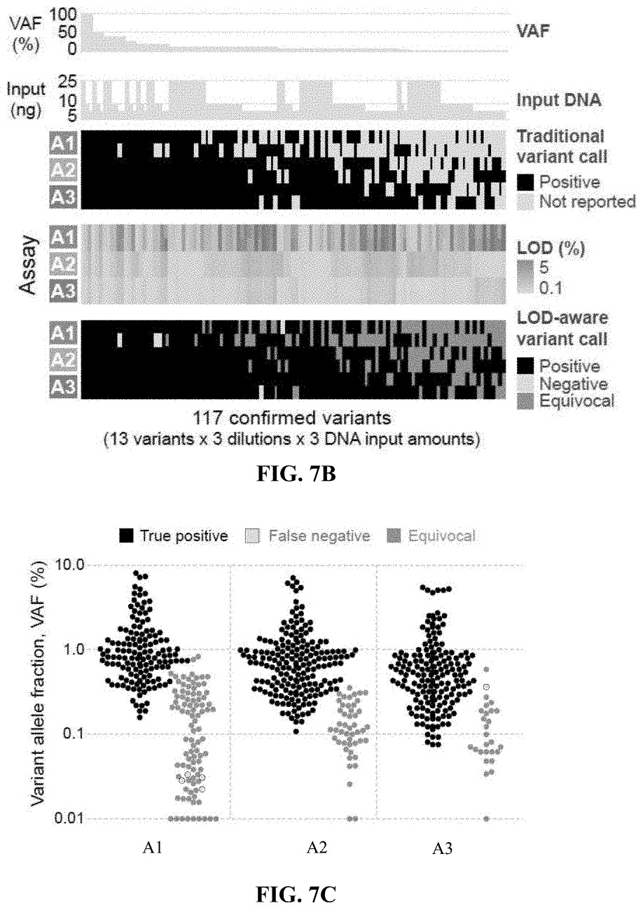

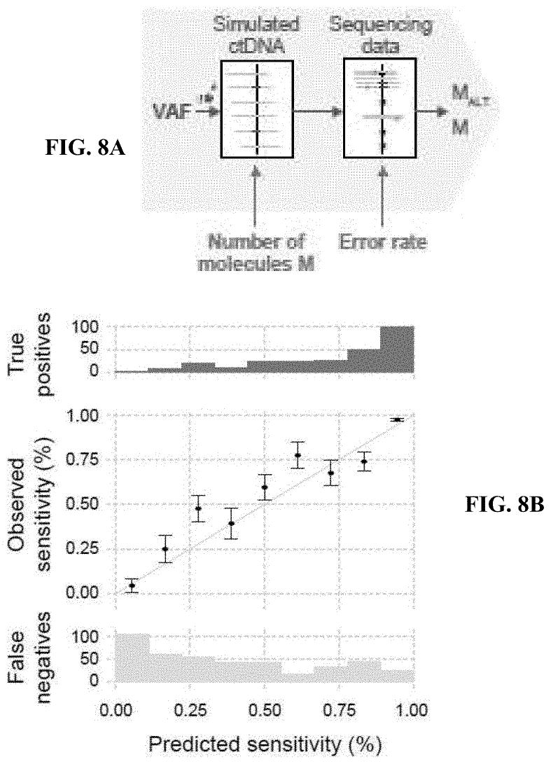

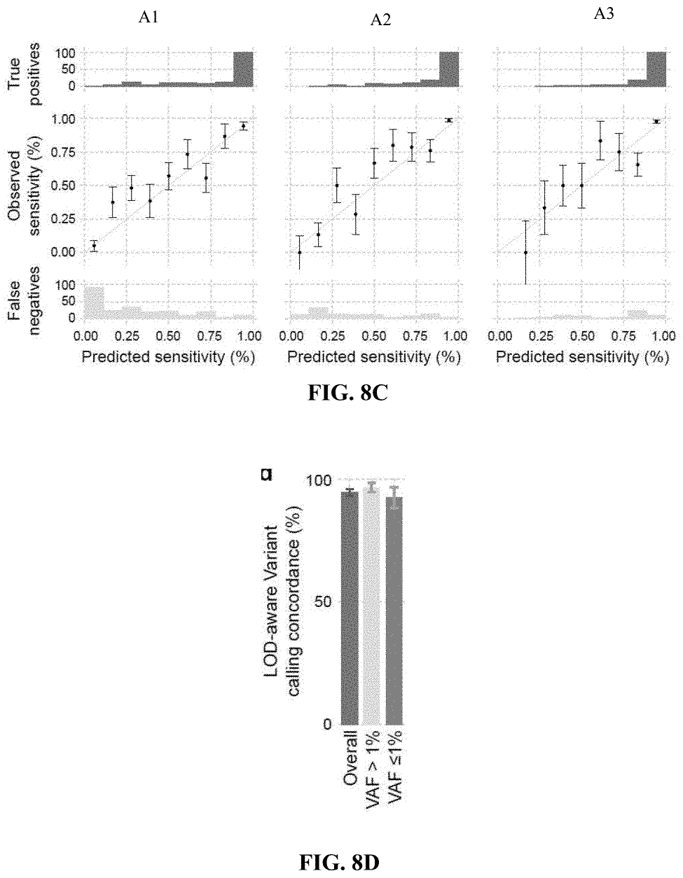

Having identified factors that may lead to false negatives and discordance between NGS assays, these factors were used to develop a variant calling framework that identifies and reports potential incorrect calls. In a standard workflow (FIG. 7A, top), sequencing data are interpreted by a variant caller that outputs positive calls but does not report positions where no variant was detected. Our “LOD-aware” framework has built on this by using an in silico generative model (FIG. 7A, encapsulated in thick dashed lines) to predict LODs for every interrogated position, which ultimately enabled positions to be triaged into positive, negative and equivocal classes (FIG. 7A, right). The inputs to this model were LCR and PCR/sequencing error rates, calculated when setting up an assay, and input amount and sequencing coverage, measured for each sample. Sequencing coverage and error rates were defined for each genomic position. The model simulated an NGS experiment in silico, following biophysical rules and incorporating stochastic sampling effects to produce synthetic sequencing data. Specifically, for each potential variant in a sample, DNA fragments were simulated for various VAFs, converted into a library and amplified by PCR. Sequencing reads were then sampled to the appropriate depth and errors injected. A variant caller processed the in-silico data to generate a curve of sensitivity vs VAF, from which the predicted LOD was read off (FIG. 7A, dark grey block arrow marked ‘LOD prediction’; LOD is defined as the lowest VAF detectable with the required sensitivity). For assay A3, the model was adjusted to reflect the inclusion of molecular barcodes (FIG. 8A).

The output from this model could then be used for LOD-aware variant calling, which takes assay limitations directly into account. Specifically, whereas standard variant callers omit positions where no variant was called, this strategy both reports these positions and assigns one of two possible labels: “equivocal”, where a high (insufficient) predicted LOD flags a potential false negative; or “negative”, where a low predicted LOD indicates we would have detected a variant if present (FIG. 7A, right). For positive calls, this approach can flag “near misses”, where a high LOD indicates this variant will not be detected in future, similar samples.

Having established an LOD-aware variant calling framework, its accuracy was checked using data from the rsctDNA experiment. Specifically, the framework was used to predict sensitivities for confirmed variants, grouped variants by these predictions, then the experimentally measured sensitivities plotted for each group (FIGS. 8B and C). The close correlation between predicted and observed sensitivities confirmed the accuracy of the above predictions and implied that the factors that were identified (FIG. 5) and included in the model sufficiently explain LOD. Next, it was tested whether this LOD-aware strategy predicts which variants were missed during variant calling for the rsctDNA data. It was assumed that variants with predicted sensitivities >50% would be detected, and remaining variants missed. This strategy correctly predicted variant calling results for 78%, 88% and 90% of the confirmed variants in assays A1, A2 and A3. It was concluded that this framework accurately predicts LOD and sensitivity and identifies when a variant caller will miss a variant.

This approach has several practical applications. First, predicted sensitivities can be used to benchmark or compare NGS assays. Second, by reporting LODs alongside variant calls and flagging potential false negatives as “equivocal”, this approach can help clinicians decide when retesting is required. Here, clinical insight is used to define a minimal VAF of interest, such as the lowest VAF proven to predict treatment response. Equivocal calls are then denoted as positions where LOD was insufficient to guarantee detection of a variant down to this threshold. To demonstrate this using the above rsctDNA dataset, the known VAFs (defined during rsctDNA preparation) were used to mimic clinical insight and provide thresholds for splitting “negative” and “equivocal” calls. Now, most calls were “positive” or “equivocal” (A1: 98.3, A2: 100%, A3: 99.6%), with just five false negatives remaining (FIG. 7). Importantly, this strategy also resolves the discordance that was observed between assays (FIG. 7B), as excluding equivocal calls now resulted in an overall concordance of 96% for VAFs≤1% (FIG. 7C and FIG. 8D). This highlights how ctDNA assay discordance primarily arises when technical capabilities are unknowingly exceeded, which can be mitigated via our LOD-aware approach.

### Example 3: LOD Predictions Improve the Reliability of Clinical NGS Tests

The clinical performance of the LOD-aware approach was evaluated as follows.

### Materials and Methods

Materials and methods as in Examples 1 and 2, and additionally:

Droplet digital PCR (ddPCR): The QX100 ddPCR system from BioRad (ddPCR, Biorad, Hercules, Calif., USA) was used, which combines a water-oil emulsion droplet technology with microfluidics (Biorad, 186-3005). All reactions were prepared using the ddPCR 2× Supermix for probes concentrated 2× (Biorad, 186-3024). The probes used targeting EGFR have previously been described (Garcia et al., 2017, supra). The ddPCR probes covered three mains somatic alterations of EGFR: various deletions of exon 19, p.L858R and p.T790M.

Digital PCR BEAMing: The EGFR p.T790M BEAMing assay was used, which is a highly sensitive and a quantitative digital PCR platform utilizing Beads, Emulsion, Amplification and Magnetics (BEAMing) provided by Sysmex Inostics (Hamburg, Germany, EU). This assay is based on a multiplex PCR targeting somatic alterations which are then followed by a massively parallel second PCR amplification performed on magnetic beads compartmentalized in millions of oil emulsions. Finally, a hybridization step with fluorescent probes specific to wild-type (WT) or mutant (MT) signals is performed by flow cytometry in order to discriminate these populations. The OncoBEAM-EGFR™ V1 kit (Sysmex Inostics, Hamburg Germany) was used, enabling only the detection of p.T790M. All experiments were performed according to the supplier's IVD recommendations for clinical applications. The pre-specified positivity threshold for each codon was established in a clinical study of 186 patients, and the clinical cut-off was defined as 50 mutant beads detected and an alternative allelic fraction superior of >0.02%.

Sensitivity analysis: In order to estimate which factor was affecting the LOD the most for a given variant in a given clinical sample, a sensitivity analysis for assays A1 and A2 was performed. Since background noise, coverage depth and input material have different units and order of magnitudes, the impact of a relative change of 10% was evaluated for each parameter. More precisely, the change in the probability of detection P_{detect} for such a parameter perturbation was calculated at the LOD. For example, for coverage N the change in the probability of detection ΔP_{N} was given by:

ΔP_{N}=P_{detect}(1.1×N,input,θ)−P_{detect}(N,input,θ) (Eq. 8)

### Results

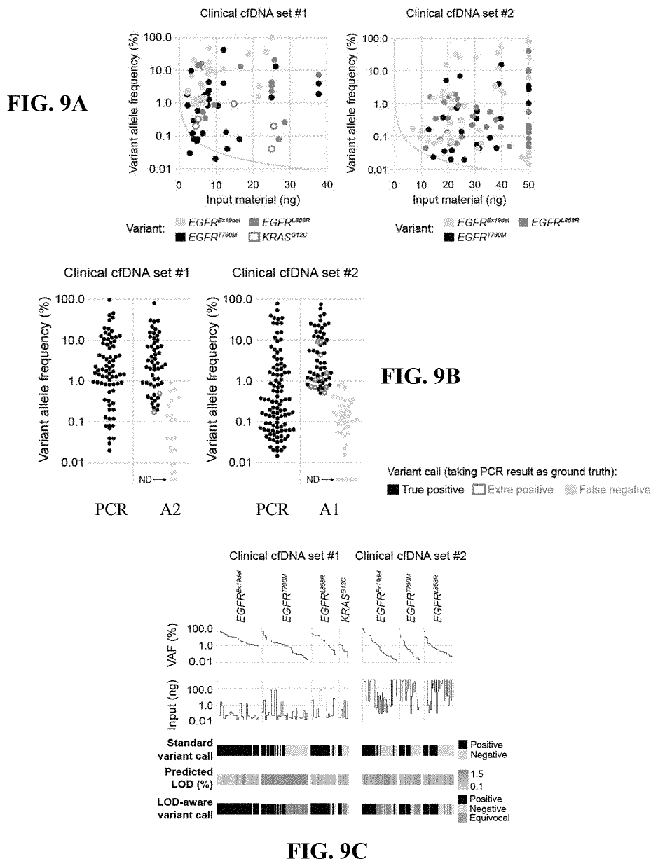

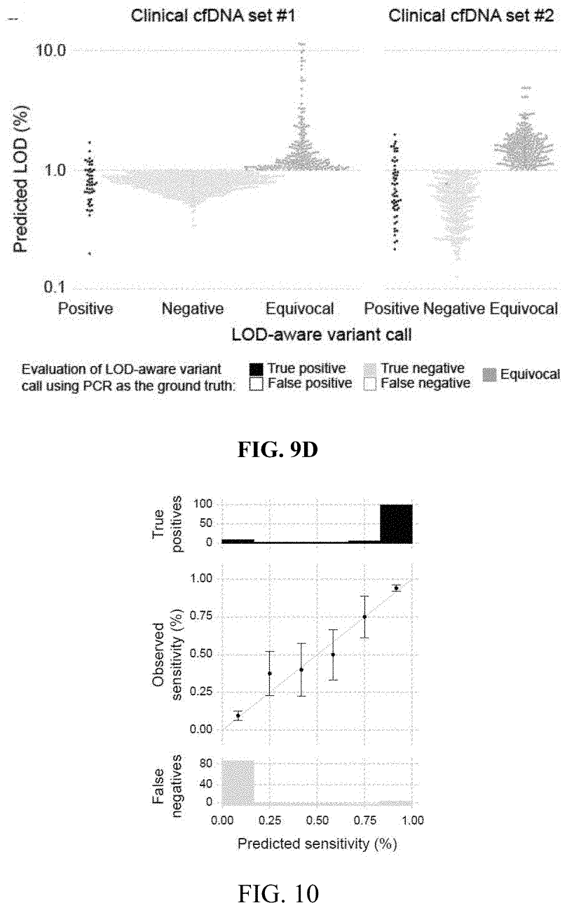

Having calibrated this LOD-aware approach using reference samples, its clinical performance was evaluated. NGS datasets from 580 NSCLC (non-small cell lung cancer) cfDNA samples were used, processed using assays A1 (n=272) and A2 (n=308). Three actionable mutations in EGFR (epidermal growth factor receptor gene) and one in KRAS (gene coding K-Ras protein) were selected, and digital PCR (dPCR, either droplet dPCR or BEAMing) used to confirm their status. This revealed 170 mutations across 126 patients, encompassing diverse VAFs and input amounts (FIG. 9A). Next, conventional variant calling was performed, and the results compared to those obtained by dPCR (FIG. 9B). It was observed that NGS assays A1 and A2 identified just 68% and 46% of the dPCR-confirmed variants, highlighting how false negatives are a major problem.

Then, it was tested whether LOD-aware variant calling solves this by disclosing potential false negatives. First, the accuracy of our model for clinical samples was confirmed, by comparing the confirmed and model predicted sensitivities for each variant (FIG. 10). Next, the standard variant calls were reclassified using our LOD-aware approach (FIG. 9C). A minimum VAF of interest of 1% was assumed, mimicking a clinical scenario in which VAFs of □1% are predictive of treatment response or have prognostic value and must be detected (Beau-Faller et al., 2020, Lung Cancer 140, 19-26). Using this threshold, negative calls with sufficient LODs (□1%) were labeled “negative”, whereas those with insufficient LODs (>1%) were reclassified as equivocal (FIG. 9D). In contrast to previous analyses, which only included cases where a confirmed variant was present, all measurements for the KRAS and three EGFR sites were plotted (i.e. confirmed positive and confirmed negative cases). Comparing our LOD-aware NGS results (excluding equivocal calls) to the dPCR “ground truth” revealed that only one false negative remained, improving inter-assay concordance from 94% to 99% for the dPCR-confirmed variants (FIG. 9D). It was concluded that LOD-aware variant calling accurately predicts the limitations of clinical ctDNA assays, foresees most false negatives and significantly improves reliability.

A unique advantage of this approach is that it is readily scalable, which was exploited to predict the ability of assays A1 and A2 to detect 89 clinically relevant variants in the NSCLC samples (FIG. 11). Here, the idea was to evaluate each site in a sample to establish whether a variant could be detected (low LOD) or was likely to be overlooked (high LOD). This uncovered a remarkably heterogeneous “LOD landscape”, with 100-fold variation between positions, samples and assays. This has reinforced the importance of reporting individual LODs for each sample and genomic positions, as enabled by our approach. Our approach also revealed the main technical factor that limits sensitivity at each position in each sample. Specifically, the change in sensitivity for a variant that is currently “just detected” (i.e. whose VAF equals the predicted LOD) was calculated for the scenario in which a model input (background noise, coverage depth or input amount) is adjusted by 10% (FIG. 11 and methods: Sensitivity analysis). This analysis was particularly informative for variants that had high (i.e. in most cases insufficient) LODs, and revealed that, for assay A1, increasing input amount led to the biggest improvement in sensitivity (and thus LOD). In contrast, increased sequencing coverage was most beneficial for assay A2.

In conclusion, our LOD-aware approach improves NGS-based variant calling by revealing LOD bottlenecks, predicting errors and increasing the transparency and reliability of variant reporting. Ultimately, this enables better informed clinical decisions. As all NGS assays are governed by the same biophysical principles, our framework is applicable to diverse applications.

### Example 4: Analysis of the Universality of LOD-Aware Variant Analysis

One advantage of the proposed methods is that they can be integrated into any genomic analysis workflow, possibly with different variant callers. The proposed computer-implemented generative model can generate data that can be used by different bioinformatics workflow algorithms to determine the LOD at specific nucleotide genomic positions. As an illustration of this, the comparison of the LOD-aware variant calling framework for rsctDNA samples between in-house and VarDict variant calling algorithms was performed as follows.

### Materials and Methods

Materials and methods as in Examples 1-3, and additionally: Variant calling with VarDict: Variants were called using VarDictJava release 1.8.2 and the recommended settings for a single sample workflow (Lai Z. et al., 2016, Nucleic Acids Res 44:e108, 2016). As VarDict does not produce a single significance score to call variants, the reported allele frequency was used as a significance score. Varying this threshold allowed to compute a ROC curve for VarDict calls on the reference standard samples. A threshold of 0.2% was fixed to obtain a similar false positive rate as for our proprietary variant caller.

LOD calculation for VarDict: To estimate the LOD of VarDict calls for Assay A2 artificial alignment files were generated using the generative model described above. For each variant interrogated 100 alignment files were produced with VAFs ranging from 0.03% to 3%. The alignment files contained paired-end reads with an insert size following a Log Normal distribution with a mean of 150 bp and a standard deviation of 57 bp, which were fitted on reference samples prepared with Assay A2. The base qualities were randomly sampled from the distribution of qualities observed in reference samples and sequencing errors were introduced at the position of interest according to background noise model. These alignments were then provided to VarDict for variant calling as described previously, producing a series of binary calls over the range of VAFs considered. A logistic regression was then fitted to these data and the LOD determined as the VAF corresponding to a probability of detection of 90%.

Analysis of BioProjetPRJNA677999 samples: Three samples were selected and prepared with Illumina TruSight Tumor 170 with UMIs and 25 ng of input from BioProjet PRJNA677999 (Jones W, et al., 2021, Genome Biol 22:111; Deveson I W, et al., 2021, Nat Biotechnol.).

FASTQ files were trimmed of their UMIs, aligned and duplex consensus counts produced as described for Assay A3. We selected 447 confirmed positive variants intersecting with the TruSight Tumor 170 panel with confirmed VAF ranging from 0.05% to 2.5%. Variant calling and LOD calculations were performed as described for Assay A3. However, as we did not have negative samples to calibrate the background noise per position on TruSight Tumor 170, we used the average of the background noise over Assay A3 panel as a constant background noise.

### Results

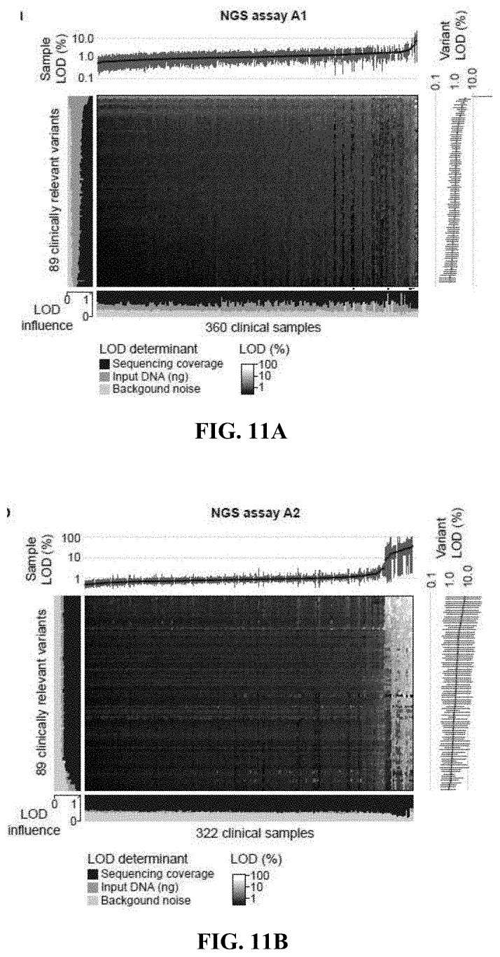

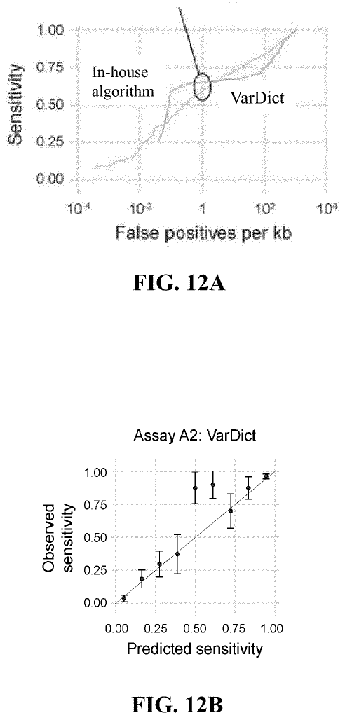

As shown above, the in-house algorithm follows best practices for ctDNA analysis (position-specific error profiles and, for A3, molecular consensuses) and its performance matched the ultra-sensitive variant caller VarDict. FIG. 12A shows the ROC analysis comparing the sensitivity and false positive rate for in-house variant calling algorithm to that of VarDict with the use of the rsctDNA data from Assay A2. For VarDict, the variant calling frequency threshold was adjusted, and for in-house algorithm, the p-value threshold was adjusted. In circle: for subsequent analyses, variant calling thresholds were set to fix the false positive rate to 1 per kb. Second, to demonstrate the validity of LOD-aware variant analysis for other variant callers, it was adopted to predict the sensitivity of VarDict for the assay A2 dataset. FIG. 12B shows the prediction of sensitivity of variant detection using VarDict to analye the rsctDNA data for assay A2 (cf FIG. 5). The same model was used as in FIG. 7A, except rather than integrating the LOD algorithm into the variant caller, synthetic bam files were generated that were presented to VarDict. The predicted sensitivities were compared to the observed sensitivities (which were used to group variants).

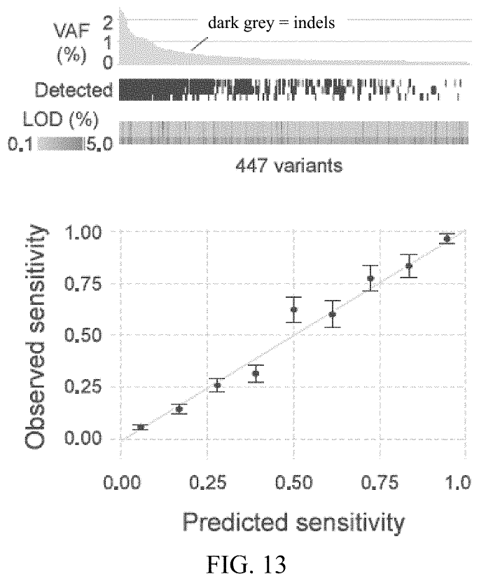

Next, the robustness and universality of LOD-aware variant analysis was evaluated. The LOD-aware variant analysis was applied to publicly available data obtained by profiling a cancer cell line reference ctDNA mixture with a barcode-equipped capture assay (Illumina) (FIG. 13) (Deveson I W, et al., supra). This dataset includes 447 germline or somatic variants (19 classified as “(Likely) Pathogenic” by ClinVar). Predicted and observed sensitivities were strongly correlated, confirming that LOD-aware variant analysis works for different assays and laboratories.

Predicted and observed sensitivities were strongly correlated, confirming that LOD-aware variant analysis works for different assays, laboratories and variant callers.

### Further Advantages and Benefits of the Proposed Methods

Prior art clinical next generation sequencing (NGS) assays are unreliable when detecting low frequency variants such as those encountered in liquid biopsies. Inclusion of methods that enable to distinguish reliable results from cases where a genetic variant may have been overlooked due to insufficient assay power can mitigate the impact of these limitations. The proposed high-throughput methods thus enable to distinguish reliable results from cases where a genetic variant may have been overlooked due to insufficient assay power. In particular, the proposed Limit of detection Aware Variant Analysis (“LAVA”) framework enables to individually predict the reliability of variant calls at each genomic position for each patient sample analyzed. The proposed computational model takes assay, sample and genomic features into account in a genomic analysis automated workflow which can interoperate with a variety of NGS assays based on their predetermined features, without the need for manual user understanding of the assay analytical factors. The proposed methods enable to estimate the reliability of each variant call to be reported to a clinician via a simple three-way classification (positive, negative or equivocal). This enables to refine false negative results and overall improves the efficiency and safety of genetic testing for routine clinical applications.

Applying the three-class, LOD-aware variant calling strategy to clinical samples demonstrated that it foresees most false negative calls and increases inter-assay concordance to □99%. The proposed approach is easily scalable, which was demonstrated by computing the “LOD landscape” for 580 non-small cell lung cancer (NSCLC) samples. This uncovered a 100-fold variation in LOD across samples and genomic positions and revealed which factors should be tuned to improve LOD. It may be concluded that high-resolution, adaptive LOD predictions improve the reliability and transparency of NGS testing, and could greatly benefit diverse applications including liquid biopsies, solid tumor analyses, personalized immunotherapy and biomarker discovery. The proposed methods and system frameworks enable this concept to be easily applied, and may thus become a central part of clinical trials, technology development and clinical decision making.

A comprehensive model of the NGS workflow accurately predicts incorrect calls, and in doing so reveals a remarkable heterogeneous LOD landscape. Variability in LOD is driven by technical factors acting at assay, sample and nucleotide levels, explaining why reporting a single global LOD (currently standard practice) leads to false negatives (Stetson et al., 2019, supra). The proposed LOD-aware variant calling methods and systems confronts this heterogeneity head on by computing position-specific LODs, answering calls for better strategies to evaluate NGS assays and report their limitations (U.S. Food and Drug Administration, 13 Apr. 2018, supra; Merker et al., 2018, supra; Tack et al., 2018, supra). As will be apparent to those skilled in the art, by assigning a third label (“equivocal”) to unreliable variant calls, false results may be more easily foreseen and managed. This dramatically improves the reproducibility (96-99% concordance) and transparency of genetic testing, ultimately leading to more efficient and reliable clinical decisions.

As will be apparent to those skilled in the art, the proposed approach has many advantages. First, it distinguishes equivocal and confident negative calls, which reduces false negatives and enables retesting to focus on essential positions, avoiding unnecessary delays. NGS tests are increasingly relying on combining data from many loci to improve sensitivity, expand the range of assayable patients or indications (Zviran et al., 2020, supra; Wan et al., 2020, Sci. Transl. Med. 12, eaaz8084; Mouliere et al., 2018, supra) or compute an aggregate score (e.g. tumor mutational burden). The proposed approach reveals which positions should be included to maximize sensitivity and minimize false negatives, which otherwise dilute the biological signal. It also “rescues” samples with low overall quality by identifying positions with sufficient sensitivity, spots near miss positive calls where LOD is borderline, and helps optimize bioinformatic thresholds to minimize false positives while maintaining the required sensitivity. Clinical trials may thus benefit from the ability of the aforementioned approach to standardize results from different study centers, patients, or timepoints, and technology development will be expedited by using an LOD-aware strategy to identify performance bottlenecks.

### Other Embodiments and Applications

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments.

While exemplary embodiments and applications of the proposed methods have been described in relation with the targeted next generation sequencing genomic analysis, it will be apparent to those skilled in the art of NGS analysis that they may also be adapted to incorporate additional features. The variant status triage could also be improved to account for the proportion of ctDNA in a cfDNA sample by using features extracted from NGS data such as DNA fragment size, nucleosome positions or mutation signatures (Jovelet et al., 2016, supra; Chabon et al., 2020, Nature 580, 245-251; Adalsteinsson et al., 2017, Nat. Commun. 8, 1324).

The Genomic Data Analyzer 120 computer system (also “system” herein) 120 may be programmed or otherwise configured to implement different genomic data analysis methods in addition to the LOD-aware variant calling systems and methods as described herein, such as receiving and/or combining sequencing data, calling copy number alterations and/or annotating variants to further characterize the tumor samples.

The proposed LOD-aware framework was validated by experiments for one of the most challenging branches of precision medicine—variant calling from ctDNA—but as the generative model within this framework is based on universal biophysical principles, this approach can benefit many NGS applications. For example, it can be easily adapted for assays detecting gene fusions, copy number alterations, epigenetic changes or nucleosome remodeling, In the context of solid tumor biopsies it can address cellular heterogeneity by revealing the lower detection limit for rare cell subpopulations (e.g. those that have acquired resistance to a treatment). More generally, as NGS technologies increasingly strive towards earlier detection of disease onset, resistance and recurrence, they will be pushed to their limits and LOD aware approaches will become indispensable.

The invention of the present disclosure may be a method for estimating a limitation of detecting a nucleic acid sequence variant (chr,pos,alt,ref) in data generated by a next-generation-sequencing (NGS) assay from a patient sample, the method comprising: obtaining alignment data, relative to a reference genome, from the patient sample NGS data; identifying from the alignment data, with a variant caller, that said variant does not have a positive call status; obtaining a measurement of a molecular count for the patient sample at a genomic position (chr,pos) of said variant; obtaining one or more analytical factors of the NGS assay used to process the patient sample; producing, with a statistical model, synthetic alignment data for one or more simulated variant allele fractions (VAF) as a function of the measured molecular count and the one or more analytical factors of the NGS assay; and estimating, from the synthetic alignment data, a detection sensitivity limitation of said variant caller as a function of one or more of the simulated VAFs, for said assay, said DNA sample and said variant (chr, pos, ref, alt).

In an embodiment, the method may further comprise obtaining a user-defined minimal variant allele fraction of interest (mVAF) for said variant; obtaining a user-defined desired sensitivity for calling said variant as positive; estimating, from the estimated detection sensitivity function, the sensitivity for said mVAF; and classifying the variant status as negative or equivocal as a function of said desired and estimated sensitivity. In a further embodiment, the method may also include obtaining a user-defined desired sensitivity for calling said variant as positive; and estimating, from the estimated detection sensitivity function, a limit of detection LODest value as the lowest simulated VAF detectable with a sensitivity larger or equal to the user-defined sensitivity.

In an embodiment, the method includes reporting the estimated limit of detection LODest value. In a further embodiment, the method includes obtaining a user-defined minimal variant allele fraction of interest (mVAF) for said variant; obtaining a user-defined desired sensitivity for calling said variant as positive; estimating, from the estimated detection sensitivity function, a limit of detection LODest value as the lowest simulated VAF detectable with a sensitivity larger or equal to the user-defined sensitivity; and wherein, if the mVAF for said variant is larger or equal to the LODest, classifying the variant status as negative, otherwise classifying the variant status as equivocal. The method may also comprise reporting the variant status.

The NGS assay may feature a molecular identifier, which may further comprise estimating the molecular count in the NGS sequencing data according to the molecular barcoding measurements in the alignment data. In an embodiment, the method further comprises measuring, in the alignment data, the total coverage at the genomic position (chr,pos) of said variant and producing, with the statistical model, the synthetic alignment data for one or more simulated VAFs as a function of the coverage.

In an embodiment, the NGS assay does not feature a molecular identifier and the analytical factors of the NGS assay comprise a library conversion rate (LCR) profile, further comprising: obtaining a DNA sample amount measurement; and estimating the molecular count in the library as a function of the DNA sample amount and the LCR value for said genomic position (chr, pos) in the LCR profile. The LCR profile may be a constant value for all genomic positions or a table of the library conversion rate value at each genomic position or set of positions. Further, the LCR profile may depend on a DNA sample type. In an embodiment, the analytical features of the NGS assay comprise an NGS assay error profile as a constant value for all variants, a table of the error rate value at each variant position (chr,pos) or set of positions, a table of the error rate value for each variant mutation type (alt,ref) or set of variant mutations, or a table of the error rate value for each variant (chr,post,ref,alt).

The NGS workflow error profile may depend on a DNA sample type. In an embodiment, wherein producing, with a statistical model, synthetic sequencing data for different simulated VAFs comprises producing for each simulated VAF value one or more BAM files or different NGS data alignment features. The statistical model may be a machine learning generative model or a biophysical generative model.

A method is proposed for estimating a limitation of detecting a nucleic acid sequence variant (chr,pos,alt,ref) in data generated by a next-generation-sequencing assay from a patient sample, the method comprising obtaining alignment data, relative to a reference genome, from the patient sample NGS data; identifying from the alignment data, with a variant caller, that said variant does not have a positive call status; obtaining a measurement of the molecular count for the patient sample at the genomic position (chr,pos) of said variant; obtaining one or more analytical factors of the NGS assay used to process the patient sample; producing, with a statistical model, synthetic alignment data for one or more simulated VAFs as a function of the measured molecular count and the analytical factors of the NGS assay; and estimating, from the synthetic alignment data, the detection sensitivity limitation of said variant caller as a function of one or more of the simulated VAFs, for said assay, said DNA sample and said variant (chr, pos, ref, alt). The synthetic data may be one or more BAM files, or one or more sets of different NGS data alignment features, for each simulated VAF value. The statistical model may be a machine learning generative model or a biophysical generative model. The NGS assay used to process the patient sample may be identified by an NGS assay identifier and the analytical factors of the NGS assay may be predetermined values stored in a memory in association with the NGS assay identifier.

In a possible embodiment, the method may further comprise obtaining a user-defined minimal variant allele fraction of interest (mVAF) for said variant; obtaining a user-defined desired sensitivity for calling said variant as positive; estimating, from the estimated detection sensitivity function, the sensitivity for said mVAF; and classifying the variant status as negative or equivocal as a function of said desired and estimated sensitivity; and reporting the variant status to an end user.

In a possible embodiment, the method may further comprise obtaining a user-defined desired sensitivity for calling said variant as positive, estimating, from the estimated detection sensitivity function, a limit of detection LODₑₛₜ value as the lowest simulated VAF detectable with a sensitivity larger or equal to the user-defined sensitivity, and reporting the estimated limit of detection LODₑₛₜ value to the user.

In a possible embodiment, the method may comprise obtaining a user-defined minimal variant allele fraction of interest (mVAF) for said variant; obtaining a user-defined desired sensitivity for calling said variant as positive; estimating, from the estimated detection sensitivity function, a limit of detection LODₑₛₜ value as the lowest simulated VAF detectable with a sensitivity larger or equal to the user-defined sensitivity; and if the mVAF for said variant is larger or equal to the LODₑₛₜ, classifying the variant status as negative, otherwise classifying the variant status as equivocal; and reporting the variant status and the LODₑₛₜ value to an end-user.

In a possible embodiment the method may estimate the molecular count in the NGS sequencing data according to molecular identifiers measurements in the alignment data.

In a possible embodiment, the method may comprise measuring, in the alignment data, the total coverage at the genomic position (chr,pos) of said variant and producing, with the statistical model, the synthetic alignment data for one or more simulated VAFs as a function of the coverage.

In a possible embodiment, the method may comprise obtaining a DNA sample amount measurement and estimating the molecular count in the library as a function of the DNA sample amount and the LCR value for the genomic position (chr, pos) in the LCR profile. In a possible embodiment, the LCR profile may be a constant value for all genomic positions. In an alternate embodiment, the LCR profile may be a table of the library conversion rate value at each genomic position, or for a set of genomic positions. In a possible embodiment, the LCR profile may depend on the DNA sample type.

In a possible embodiment, the analytical features of the NGS assay may comprise an NGS assay error profile as a constant value for all variants, or as a table of the error rate value at each variant position (chr,pos) or set of positions, or as a table of the error rate value for each variant mutation type (alt,ref) or set of variant mutations, or as a table of the error rate value for each variant (chr,post,ref,alt). In a possible embodiment, the NGS workflow error profile may depend on a DNA sample type.

In summary, the proposed systems and methods pave the way to a new standard in variant calling and offers a much-needed improvement in the reliability of NGS-based clinical testing.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As will be apparent to those skilled in the art of digital data communications, the methods described herein may be indifferently applied to various data structures such as data files or data streams. The terms “data”, “data set”, “data structures”, “data fields”, “file”, or “stream” may thus be used indifferently throughout this specification.

Although the detailed description above contains many specific details, these should not be construed as limiting the scope of the embodiments but as merely providing illustrations of some of several embodiments.

In addition, it should be understood that any figures which highlight the functionality and advantages are presented for example purposes only. The disclosed methods are sufficiently flexible and configurable such that they may be utilized in ways other than that shown.

What is claimed is:

1. A method for estimating a limit of detection (LOD) when characterizing a nucleic acid sequence variant (chr,pos,alt,ref) in data generated by a next-generation-sequencing (NGS) assay from a patient sample, the method comprising:
obtaining alignment data, relative to a reference genome, from the patient sample NGS data;
identifying from the alignment data, with a variant caller, that said variant does not have a positive call status;
obtaining a measurement of the molecular count for the patient sample at the genomic position (chr,pos) of said variant;
obtaining one or more analytical factors of the NGS assay used to process the patient sample;
producing, with a statistical model, synthetic alignment data for one or more simulated VAFs as a function of the measured molecular count and the analytical factors of the NGS assay; and
estimating, from the synthetic alignment data, the detection sensitivity of said variant caller as a function of one or more of the simulated VAFs, for said assay, said DNA sample and said variant (chr, pos, ref, alt).

2. The method of claim 1, wherein the NGS assay used to process the patient sample is identified by an NGS assay identifier and wherein the one or more analytical factors of the NGS assay are predetermined values stored in a memory in association with the NGS assay identifier.

3. The method of claim 1, further comprising:
obtaining a user-defined minimal variant allele fraction of interest (mVAF) for said variant;
obtaining a user-defined desired sensitivity for calling said variant as positive;
estimating, from the estimated detection sensitivity function, the sensitivity for said mVAF; and
classifying the variant status as negative or equivocal as a function of said desired and estimated sensitivity.

4. The method of claim 2, further comprising:
obtaining a user-defined minimal variant allele fraction of interest (mVAF) for said variant;
obtaining a user-defined desired sensitivity for calling said variant as positive;
estimating, from the estimated detection sensitivity function, the sensitivity for said mVAF; and
classifying the variant status as negative or equivocal as a function of said desired and estimated sensitivity.

5. The method of claim 1, further comprising:
obtaining a user-defined desired sensitivity for calling said variant as positive; and
estimating, from the estimated detection sensitivity function, a limit of detection LODₑₛₜ value as the lowest simulated VAF detectable with a sensitivity larger or equal to the user-defined sensitivity.

6. The method of claim 2, further comprising:
obtaining a user-defined desired sensitivity for calling said variant as positive; and
estimating, from the estimated detection sensitivity function, a limit of detection LODₑₛₜ value as the lowest simulated VAF detectable with a sensitivity larger or equal to the user-defined sensitivity.

7. The method of claim 5, further comprising reporting the estimated limit of detection LODₑₛₜ value.

8. The method of claim 1, further comprising:
obtaining a user-defined minimal variant allele fraction of interest (mVAF) for said variant;
obtaining a user-defined desired sensitivity for calling said variant as positive;
estimating, from the estimated detection sensitivity function, a limit of detection LODₑₛₜ value as the lowest simulated VAF detectable with a sensitivity larger or equal to the user-defined sensitivity; and
if the mVAF for said variant is larger or equal to the LODₑₛₜ, classifying the variant status as negative, otherwise classifying the variant status as equivocal.

9. The method of claim 2, further comprising:
obtaining a user-defined minimal variant allele fraction of interest (mVAF) for said variant;
obtaining a user-defined desired sensitivity for calling said variant as positive;
estimating, from the estimated detection sensitivity function, a limit of detection LODₑₛₜ value as the lowest simulated VAF detectable with a sensitivity larger or equal to the user-defined sensitivity; and
if the mVAF for said variant is larger or equal to the LODₑₛₜ, classifying the variant status as negative, otherwise classifying the variant status as equivocal.

10. The method of claim 3, further comprising reporting the variant status.

11. The method of claim 8, further comprising reporting the variant status.

12. The method of claim 1, wherein the NGS assay features a molecular identifier, further comprising estimating the molecular count in the NGS sequencing data according to a molecular barcoding measurements in the alignment data.

13. The method of claim 1, further comprising measuring, in the alignment data, a total coverage at the genomic position (chr,pos) of said variant and producing, with the statistical model, the synthetic alignment data for one or more simulated VAFs as a function of the coverage.

14. The method of claim 1, wherein the NGS assay does not feature a molecular identifier and the analytical factors of the NGS assay comprise a library conversion rate (LCR) profile, further comprising:
obtaining a DNA sample amount measurement; and
estimating the molecular count in the library as a function of the DNA sample amount and a LCR value for said genomic position (chr, pos) in the LCR profile.

15. The method of claim 14, wherein the LCR profile is selected from the group consisting of a constant value for all genomic positions or a table of the library conversion rate value at each genomic position or set of positions.

16. The method of claim 14 wherein the LCR profile depends on a DNA sample type.

17. The method of claim 1, wherein the analytical features of the NGS assay comprise an NGS assay error profile selected from the group consisting of a constant value for all variants, a table of the error rate value at each variant position (chr,pos) or set of positions, a table of the error rate value for each variant mutation type (alt,ref) or set of variant mutations, or a table of the error rate value for each variant (chr,post,ref,alt).

18. The method of claim 17, wherein the NGS assay error profile depends on a DNA sample type.

19. The method of claim 1, wherein producing, with a statistical model, synthetic sequencing data for different simulated VAFs comprises producing for each simulated VAF value one or more BAM files or different NGS data alignment features.

20. The method of claim 1, wherein the statistical model is selected from the group consisting of a machine learning generative model or a biophysical generative model.
